(19) United States
(12) Patent Application Publication
(10) Pub. No.: US 2025/0090435 A1
(43) Pub. Date: Mar. 20, 2025

(54) PRESERVATIVES FOR COSMETIC AND PHARMACEUTICAL COMPOSITIONS

(71) Applicant: SACHEM, INC., Austin, TX (US)

(72) Inventors: Helénè Eeckman, Brakel (BE); Tim Engel, Tilburg (NL); Cedrick Mahieux, Austin, TX (US); Ari Kar, Austin, TX (US); Robert York, Austin, TX (US)

(73) Assignee: SACHEM, INC., Austin, TX (US)

(21) Appl. No.: 18/291,721

(22) PCT Filed: Jul. 29, 2022
(86) PCT No.: PCT/US2022/038929

§ 371 Date: Jan. 24, 2024

Related U.S. Application Data
(60) Provisional application No. 63/226,862, filed on Jul. 29, 2021.

(51) Int. Cl. A61K 8/34; A61K 8/44; A61Q 5/02; A61Q 19/00

(52) U.S. Cl. CPC A61K 8/345; A61K 8/44; A61Q 5/02; A61Q 19/00; A61K 2800/10; A61K 2800/524

(57) ABSTRACT

Various preservatives and compositions incorporating these preservatives as well as various methods of using these preservatives and compositions are described. For example, various compositions that include cosmetic and pharmaceutical compositions containing a preservative such as 3-(heptyloxy)propane-1,2-diol and one or more other ingredients (e.g., natural ingredients) are described.

### FIELD OF THE INVENTION

The present invention relates to various preservatives and compositions incorporating these preservatives as well as various methods of using these preservatives and compositions. For example, various compositions of the present invention include cosmetic and pharmaceutical compositions containing a preservative such as the naturally derived glyceryl ether 3-(heptyloxy)propane-1,2-diol and one or more other ingredients (e.g., natural ingredients).

### BACKGROUND OF THE INVENTION

Various compositions, particularly compositions for personal care (e.g., pharmaceutical and cosmetic compositions) can be contaminated with naturally occurring microorganisms such as fungi and bacteria. Contamination can result from direct contact of the composition with the user's skin (e.g., deodorant, lipstick, etc.) or during the preparation of the compositions (e.g. water, air, etc.). Contamination can also result from the use/repeated use of implements or applicators such as brushes, wipes, cotton pads, etc. that are used to apply the compositions to a user's skin. Preservatives can be added to these compositions to enhance long-term stability by providing the capacity to eliminate introduced microorganisms and prevent their permanent colonization of the compositions. However, these preservatives must be at a relatively high enough concentration to have antimicrobial efficacy without being excessively high as to cause harm or irritation to the user or to exceed regulatory limits.

Further, high concentrations of preservatives can negatively affect formulation properties. For example, compositions having a high concentration of one or more preservatives can exhibit reduced formulation stability and undesirable rheological properties. Thus, there remains a need for preservatives that are effective at low concentrations, that cause less harm or irritation, and/or do not negatively affect formulation properties.

### BRIEF SUMMARY OF THE INVENTION

Various aspects of the present invention are directed to compositions (e.g., cosmetic and pharmaceutical compositions) comprising 3-(heptyloxy)propane-1,2-diol and at least one additional ingredient.

Further aspects relate to methods of preserving and/or enhancing the rheology of (thickening) a cosmetic or pharmaceutical composition. The methods comprise mixing 3-(heptyloxy)propane-1,2-diol with at least one additional ingredient of the composition.

Other aspects relate to methods of cleansing, treating, or caring for skin, hair, or teeth in a subject in need thereof. The methods comprise applying compositions of the first aspect to the skin, hair, or teeth of the subject.

Other objects and features will be in part apparent and in part pointed out hereinafter.

### BRIEF DESCRIPTION OF THE DRAWINGS

FIG. 1 depicts viscosity increase in sulfated surfactant systems with APG (i.e. alkyl polyglucoside) as detailed in Example 5. Each column represents a formulation for combinations of sulfated surfactant systems (Na Coco Sulfate, Ammonium Lauryl Sulfate, and Na Lauryl Ether Sulfate) and preservatives (no preservative, heptyloxy (i.e. 3-(heptyloxy)propane-1,2-diol), octyloxy (i.e. 3-[(n-octyl)oxy]-1,2-propanediol), and heptyloxy+octyloxy (i.e. 3-(heptyloxy)propane-1,2-diol+3-[(n-octyl)oxy]-1,2-propanediol).

FIG. 2 depicts viscosity increase in sulfated surfactant systems with CAPB (i.e. Cocamidopropyl betaine) as detailed in Example 5. Each column represents a formulation for combinations of sulfated surfactant systems (Ammonium Lauryl Sulfate and Na Lauryl Ether Sulfate) and preservatives (no preservative, heptyloxy (i.e. 3-(heptyloxy)propane-1,2-diol), octyloxy (i.e. 3-[(n-octyl)oxy]-1,2-propanediol), and heptyloxy+octyloxy (i.e. 3-(heptyloxy)propane-1,2-diol+3-[(n-octyl)oxy]-1,2-propanediol).

FIG. 3 depicts viscosity increase in salted and unsalted formulations as detailed in Example 5. Each column represents a formulation for combinations of preservatives (Formulation (i.e. no preservative), Formulation+heptyloxy (i.e. 3-(heptyloxy)propane-1,2-diol), and Formulation+heptyloxy+octyloxy (i.e. 3-(heptyloxy)propane-1,2-diol+3-[(n-octyl)oxy]-1,2-propanediol) and salt conditions (with and without sodium chloride).

FIG. 4A depicts a shampoo formulation made with 3-(heptyloxy)propane-1,2-diol and 3-[(n-octyl)oxy]-1,2-propanediol added in sequence to the formulation, which results in a precipitate as described in Example 6.

FIG. 4B depicts a shampoo formulation made with 3-(heptyloxy)propane-1,2-diol and 3-[(n-octyl)oxy]-1,2-propanediol being premixed together before being added to the formulation, which results in a clear solution as described in Example 6.

### DETAILED DESCRIPTION OF THE INVENTION

The present invention relates to various preservatives and compositions incorporating these preservatives as well as various methods of using these preservatives and compositions. For example, various compositions of the present invention include cosmetic and pharmaceutical compositions containing a preservative such as 3-(heptyloxy)propane-1,2-diol and one or more other ingredients (e.g., natural ingredients). As used herein, the term “preservative” includes ingredients which are added with the primary or exclusive purpose of inhibiting microbial growth (e.g., an antimicrobial) and/or preventing undesirable chemical changes in the composition.

It has been surprisingly discovered that 3-(heptyloxy)propane-1,2-diol blocks microbial growth in compositions (e.g., cosmetic and pharmaceutical compositions) with broad efficacy over a wide pH range, even when used at low concentrations. Further, 3-(heptyloxy)propane-1,2-diol has been found to exhibit reduced/no skin irritancy, and can be used as a non-skin sensitizer. In addition, it has been discovered that 3-(heptyloxy)propane-1,2-diol is a multi-functional ingredient. For example, this ingredient functions as a thickener/rheology modifier and as a surfactant/emulsifier in various compositions such as shampoo (e.g., all-natural shampoo compositions), natural wet wipes, emulsion wet wipes, and roll-on deodorant. This ingredient can provide for enhanced formulation capabilities, efficacy, hydration/humectant effect, moisturizing effect, sensorial effect, and deo-effect as compared to current preservatives. Advantageously, 3-(heptyloxy)propane-1,2-diol provides for compositions requiring fewer additives as compared to existing compositions. As such, the preservative and preservative combinations described herein provide for improved anti-microbial efficacy and enhanced formulation capabilities especially for all-natural, aqueous compositions. In some embodiments, the composition is at least 95% natural according to ISO standard 16128.

Various aspects of the present invention are directed to a high purity concentrate composition comprising 3-(heptyloxy)propane-1,2-diol. For example, the concentrate composition can have a concentration of 3-(heptyloxy)propane-1,2-diol that is about 90 wt. % or greater, about 91 wt. % or greater, about 92 wt. % or greater, about 93 wt. % or greater, about 94 wt. % or greater, about 95 wt. % or greater, about 96 wt. % or greater, about 97 wt. % or greater, about 98 wt. % or greater, about 99 wt. % or greater, about 99.9 wt. % or greater, or about 99.99 wt. % or greater. The concentrate composition is useful for the preparation and preservation of various other downstream compositions as described herein. The concentrate composition can further comprise one or more additional ingredients as described herein.

Further aspects of the present invention are directed to compositions comprising 3-(heptyloxy)propane-1,2-diol and at least one additional ingredient. These compositions can be, for example, various types of personal care compositions. The compositions can be cosmetic and/or pharmaceutical compositions.

In various embodiments, the composition comprises an aqueous composition. The composition can be a solution, emulsion, gel, or suspension. In some embodiments, the composition is a gel and the gel can an oil-in-water (o/w) gel, cream gel, hydro-alcoholic gel, or hydrogel. In certain embodiments, the composition is the aqueous phase of an oil-in-water emulsion or a water-in-oil emulsion.

In various embodiments, the composition is a topical composition (i.e., a composition suitable for topical or application to the external surfaces of a user.

Also, the composition can be a leave-on composition (e.g., deodorant) or a rinse-off composition (e.g., shampoo).

Examples of particular cosmetic and pharmaceutical compositions include shampoo, conditioner, dry shampoo, body wash, facial cleanser, face mask, bubble bath, cleansing milk, micellar water, make-up remover, cleansing wipes, baby wipes, hair mask, liquid soap, shaving soap, shaving foam, cleansing foam, day cream, anti-aging cream, body milk, facial lotion, body lotion, in-shower body lotion, body mousse, face serum, eye cream, sunscreen lotion, sun cream, face cream, after-shave lotion, pre-shaving cream, depilatory cream, skin-whitening gel, self-tanning cream, anti-acne gel, hair oil, hair styling gel, hair styling cream, pomade, anti-frizz serum, detangler, scalp treatment, hair colorant, split end fluid, deodorant, antiperspirant, baby cream, insect repellent, hand cream, sunscreen lotion, sunscreen gel, foot cream, exfoliator, body scrub, bar soap, hair treatment, mouthwash, toothpaste, teeth whitener, moisturizer, serum, toner, aqua sorbet, cream gel, styling mousse, hydro-alcoholic gel, body oil, shower milk, hair spray, combing cream, sunblock, fragrance, perfume, mascara, foundation, primer, concealer, blush, bronzer, setting powder, setting spray, blemish balm cream, color correcting cream, eyeliner, brow liner, night cream, eye brow gel, highlighter, lip stain, lip stick, lip gloss, lip balm, lip crayon, hand sanitizer, nail varnish remover, cellulite treatment, cuticle cream, eye shadow, bath additive, body mist, eau de toilette, lubricating gel, illuminator, hair spray, combing cream, baby powder, and diaper rash cream.

In certain embodiments, the composition is selected from the group consisting of shampoo, conditioner, body wash, facial cleanser, and bubble bath.

In various embodiments, the composition comprises a medicated composition containing at least one medicament.

In some embodiments, the composition is loaded onto a solid substrate. The solid substrate can comprise a wipe (wet-wipe), pad, tissue, sponge, or towel.

In general, the concentration of 3-(heptyloxy)propane-1,2-diol is sufficient to provide for an antimicrobial effect either alone or in combination with one or more other preservatives. For example, the composition can comprise a concentration of 3-(heptyloxy)propane-1,2-diol that is about 0.25 wt. % or greater, about 0.5 wt. % or greater, or about 1 wt. % or greater. In various embodiments, the composition comprises a concentration of 3-(heptyloxy)propane-1,2-diol that is from about 0.25 wt. % to about 5 wt. %, from about 0.25 wt. % to about 3 wt. %, from about 0.25 wt. % to about 1.5 wt. %, from about 0.25 wt. % to about 1 wt. %, from about 0.5 wt. % to about 5 wt. %, from about 0.5 wt. % to about 3 wt. %, from about 0.5 wt. % to about 1.5 wt. %, from about 0.5 wt. % to about 1 wt. %, from about 1 wt. % to about 5 wt. %, from about 1 wt. % to about 3 wt. %, or from about 1 wt. % to about 1.5 wt. %. In some embodiments, the composition comprises a concentration of 3-(heptyloxy)propane-1,2-diol that is about 0.01 wt. % or greater, about 0.1 wt. % or greater, about 0.25 wt. % or greater, about 0.5 wt. % or greater, or about 1 wt. % or greater. In some embodiments, the composition comprises a concentration of 3-(heptyloxy)propane-1,2-diol that is from about 0.1 wt. % to about 10 wt. %, from about 0.1 wt. % to about 3 wt. %, from about 0.1 wt. % to about 1.5 wt. %, from about 0.1 wt. % to about 1 wt. %, from about 0.25 wt. % to about 5 wt. %, from about 0.25 wt. % to about 3 wt. %, from about 0.25 wt. % to about 1.5 wt. %, from about 0.25 wt. % to about 1 wt. %, from about 0.5 wt. % to about 5 wt. %, from about 0.5 wt. % to about 3 wt. %, from about 0.5 wt. % to about 1.5 wt. %, from about 0.5 wt. % to about 1 wt. %, from about 1 wt. % to about 5 wt. %, from about 1 wt. % to about 3 wt. %, or from about 1 wt. % to about 1.5 wt. %.

As noted, the 3-(heptyloxy)propane-1,2-diol preservative can be used in compositions over a broad pH range. For example, the composition can have a pH of about 10.5 or less, about 9 or less, about 8 or less, or about 7 or less. Additionally or alternatively, the composition can have a pH of about 2 or greater, about 3 or greater, or about 4 or greater.

In various embodiments, the composition has a pH of from about 2 to about 10.5, from about 3 to about 8, from about 4 to about 7, from about 4 to about 6, from about 5 to about 7, from about 5.5 to about 7, from about 5.5 to about 6.5, from about 5 to about 6, or about 5.5.

### Additional Ingredients—Other Preservatives

The composition can further comprises at least one other preservative (i.e., in addition to 3-(heptyloxy)propane-1,2-diol). For example, the composition can comprise at least one other preservative (i.e., in addition to 3-(heptyloxy)propane-1,2-diol) at a concentration that is about 1 wt. % or greater, about 2 wt. % or greater, about 5 wt. % or greater, about 10 wt. % or greater, about 15 wt. % or greater, or about 20 wt. % or greater. In some embodiments, the concentration of the at least one other preservative can be from about 1 wt. % to about 50 wt. %, from about 2 wt. % to about 50 wt. %, from about 5 wt. % to about 50 wt. %, from about 10 wt. % to about 50 wt. %, from about 20 wt. % to about 50 wt. %, from about 1 wt. % to about 25 wt. %, from about 2 wt. % to about 25 wt. %, from about 5 wt. % to about 25 wt. %, from about 10 wt. % to about 25 wt. %, from about 1 wt. % to about 15 wt. %, from about 2 wt. % to about 15 wt. %, from about 5 wt. % to about 15 wt. %, from about 10 wt. % to about 15 wt. %, from about 1 wt. % to about 10 wt. %, from about 2 wt. % to about 10 wt. %, from about 5 wt. % to about 10 wt. %, or from about 1 wt. % to about 5 wt. %.

In various embodiments, the weight ratio of 3-(heptyloxy)propane-1,2-diol to the additional preservative can be from about 1:20 to about 20:1, from about 1:10 to about 10:1, from about 5:1 to about 1:5, or about 1:1. Further, the amount of 3-(heptyloxy)propane-1,2-diol can be in excess relative to the additional preservative. For example, the weight ratio of 3-(heptyloxy)propane-1,2-diol to the additional preservative can be about 1.1:1 or greater, about 1.5:1 or greater, about 2:1 or greater, about 3:1 or greater, about 4:1 or greater, about 5:1 or greater, about 10:1 or greater, or about 25:1 or greater. In some embodiments, the weight ratio of 3-(heptyloxy)propane-1,2-diol to the additional preservative is from about 1:1 to about 100:1, from about 1:1 to about 50:1, from about 1:1 to about 20:1, from about 1.1:1 to about 100:1, from about 1.1:1 to about 50:1, from about 1.1:1 to about 20:1, from about 2:1 to about 100:1, from about 2:1 to about 50:1, from about 2:1 to about 20:1, from about 5:1 to about 100:1, from about 5:1 to about 50:1, from about 5:1 to about 20:1, from about 10:1 to about 100:1, from about 10:1 to about 50:1, from about 10:1 to about 20:1, from about 20:1 to about 100:1, from about 20:1 to about 50:1, or from about 50:1 to about 100:1. In some embodiments, the weight ratio of the additional preservative to the 3-(heptyloxy)propane-1,2-diol is from about 1:1 to about 100:1, from about 1:1 to about 50:1, from about 1:1 to about 20:1, from about 1.1:1 to about 100:1, from about 1.1:1 to about 50:1, from about 1.1:1 to about 20:1, from about 2:1 to about 100:1, from about 2:1 to about 50:1, from about 2:1 to about 20:1, from about 5:1 to about 100:1, from about 5:1 to about 50:1, from about 5:1 to about 20:1, from about 10:1 to about 100:1, from about 10:1 to about 50:1, from about 10:1 to about 20:1, from about 20:1 to about 100:1, from about 20:1 to about 50:1, or from about 50:1 to about 100:1.

In some embodiments, the 3-(heptyloxy)propane-1,2-diol constitutes about 10 wt. % or greater, about 20 wt. % or greater, about 30 wt. % or greater, about 40 wt. % or greater, about 50 wt. % or greater, about 60 wt. % or greater, about 70 wt. % or greater, about 80 wt. % or greater, about 90 wt. % or greater, or about 95 wt. % or greater of the total preservative content present in the composition. In certain embodiments, the 3-(heptyloxy)propane-1,2-diol constitutes from about 10 wt. % to about 99 wt. %, from about 30 wt. % to about 99 wt. %, from about 50 wt. % to about 99 wt. %, from about 70 wt. % to about 99 wt. %, from about 90 wt. % to about 99 wt. %, from about 10 wt. % to about 95 wt. %, from about 30 wt. % to about 95 wt. %, from about 50 wt. % to about 95 wt. %, from about 70 wt. % to about 95 wt. %, from about 90 wt. % to about 95 wt. %, from about 10 wt. % to about 90 wt. %, from about 30 wt. % to about 90 wt. %, from about 50 wt. % to about 90 wt. %, from about 70 wt. % to about 90 wt. %, from about 10 wt. % to about 80 wt. %, from about 30 wt. % to about 80 wt. %, from about 50 wt. % to about 80 wt. %, from about 70 wt. % to about 80 wt. %, from about 10 wt. % to about 70 wt. %, from about 30 wt. % to about 70 wt. %, or from about 50 wt. % to about 70 wt. % of the total preservative content present in the composition.

In various embodiments, the additional ingredient comprises a glycerol derivative, which can also function as a preservative. The glycerol derivative can be a compound of formula (I):

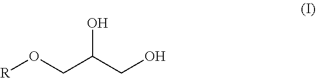

wherein R is optionally substituted C₃-C₁₈ alkyl, optionally substituted C₃-C₁₈ alkenyl, or optionally substituted C₆-C₁₀ aryl.

In some embodiments, R is C₃-C₁₈ alkyl or C₃-C₁₈ alkenyl that is branched or unbranched, and is substituted by one or more hydroxyl groups, one or more C₁-C₄ alkoxy groups, or both one or more hydroxyl and one or more C₁-C₄ alkoxy groups. In certain embodiments, R is C₃-C₁₈ alkyl, whether branched or unbranched, is interrupted by up to four oxygen atoms within the alkyl chain. In further embodiments, R is a C₆-C₁₀ aryl that is substituted by one or more hydroxyl, one or more C₁-C₄ alkoxy groups, or both one or more hydroxyl and one or more C₁-C₄ alkoxy groups.

The term “alkyl”, as used herein, refers to saturated monovalent hydrocarbyl groups that can be straight or branched, cyclic or acyclic moieties, such as but not limited to, methyl, ethyl, propyl, butyl, pentyl, hexyl, octyl groups, etc. and corresponding cyclic moieties thereof (e.g., cyclopropyl, cyclobutyl, cyclopentyl and so on).

The term “alkenyl”, as used herein refers to acyclic branched or unbranched hydrocarbons having one carbon-carbon double bond. As such, an alkenyl can be partially saturated or unsaturated.

The term “aryl”, as used herein, refers to a carbocyclic aromatic group. Examples of aryl groups include, but are not limited to, phenyl, benzyl, naphthyl, or anthracenyl.

The term “alkoxy”, as used herein, includes O-alkyl groups wherein alkyl is as defined above and O represents oxygen. Representative alkoxy groups include, but are not limited to, —O-methyl, —O-ethyl, —O-n-propyl, —O-n-butyl. An alkoxy can be saturated, partially saturated, or unsaturated.

In various embodiments, the additional ingredient (or additional preservative) comprises at least one glycerol derivative selected from the group consisting of 3-[(2-ethylhexyl)oxy]-1,2-propanediol, 3-[(n-octyl)oxy]-1,2-propanediol, and 3-[(2-octyl)oxy]-1,2-propanediol. In some embodiments, the additional ingredient comprises at least one glycerol derivative selected from the group consisting of (iso-)butyl glycerylether, (iso-)amylglycerylether, hexylglycerylether, methylheptylglycerylether, 3-octylglycerylether, 1-nonylglycerylether, and 1-dodecylglycerylether.

In various embodiments, the additional ingredient (or additional preservative) comprises 3-[(2-octyl)oxy]-1,2-propanediol. In various embodiments, the composition comprises a concentration of 3-[(n-octyl)oxy]-1,2-propanediol that is about 0.01 wt. % or greater, about 0.1 wt. % or greater, about 0.25 wt. % or greater, about 0.5 wt. % or greater, or about 1 wt. % or greater. In various embodiments, the composition comprises a concentration of 3-[(n-octyl)oxy]-1,2-propanediol that is from about 0.1 wt. % to about 10 wt. %, from about 0.1 wt. % to about 3 wt. %, from about 0.1 wt. % to about 1.5 wt. %, from about 0.1 wt. % to about 1 wt. %, from about 0.25 wt. % to about 5 wt. %, from about 0.25 wt. % to about 3 wt. %, from about 0.25 wt. % to about 1.5 wt. %, from about 0.25 wt. % to about 1 wt. %, from about 0.5 wt. % to about 5 wt. %, from about 0.5 wt. % to about 3 wt. %, from about 0.5 wt. % to about 1.5 wt. %, from about 0.5 wt. % to about 1 wt. %, from about 1 wt. % to about 5 wt. %, from about 1 wt. % to about 3 wt. %, or from about 1 wt. % to about 1.5 wt. %.

The composition can comprise the at least one glycerol derivative at a concentration that is about 1 wt. % or greater, about 2 wt. % or greater, about 5 wt. % or greater, about 10 wt. % or greater, about 15 wt. % or greater, or about 20 wt. % or greater. In some embodiments, the concentration of the at least one glycerol derivative can be from about 1 wt. % to about 50 wt. %, from about 2 wt. % to about 50 wt. %, from about 5 wt. % to about 50 wt. %, from about 10 wt. % to about 50 wt. %, from about 20 wt. % to about 50 wt. %, from about 1 wt. % to about 25 wt. %, from about 2 wt. % to about 25 wt. %, from about 5 wt. % to about 25 wt. %, from about 10 wt. % to about 25 wt. %, from about 1 wt. % to about 15 wt. %, from about 2 wt. % to about 15 wt. %, from about 5 wt. % to about 15 wt. %, from about 10 wt. % to about 15 wt. %, from about 1 wt. % to about 10 wt. %, from about 2 wt. % to about 10 wt. %, from about 5 wt. % to about 10 wt. %, or from about 1 wt. % to about 5 wt. %.

In various embodiments, the weight ratio of 3-(heptyloxy)propane-1,2-diol to the at least one glycerol derivative can be from about 1:20 to about 20:1, from about 1:10 to about 10:1, from about 5:1 to about 1:5, or about 1:1. Further, the amount of 3-(heptyloxy)propane-1,2-diol can be in excess relative to the at least one glycerol derivative. For example, the weight ratio of 3-(heptyloxy)propane-1,2-diol to the at least one glycerol derivative can be about 1.1:1 or greater, about 1.5:1 or greater, about 2:1 or greater, about 3:1 or greater, about 4:1 or greater, about 5:1 or greater, about 10:1 or greater, or about 25:1 or greater. In some embodiments, the weight ratio of 3-(heptyloxy)propane-1,2-diol to the at least one glycerol derivative is from about 1:1 to about 100:1, from about 1:1 to about 50:1, from about 1:1 to about 20:1, from about 1.1:1 to about 100:1, from about 1.1:1 to about 50:1, from about 1.1:1 to about 20:1, from about 2:1 to about 100:1, from about 2:1 to about 50:1, from about 2:1 to about 20:1, from about 5:1 to about 100:1, from about 5:1 to about 50:1, from about 5:1 to about 20:1, from about 10:1 to about 100:1, from about 10:1 to about 50:1, from about 10:1 to about 20:1, from about 20:1 to about 100:1, from about 20:1 to about 50:1, or from about 50:1 to about 100:1.

In various embodiments, 3-(heptyloxy)propane-1,2-diol constitutes about 10 wt. % or greater, about 20 wt. % or greater, about 30 wt. % or greater, about 40 wt. % or greater, about 50 wt. % or greater, about 60 wt. % or greater, about 70 wt. % or greater, about 80 wt. % or greater, about 90 wt. % or greater, or about 95 wt. % or greater of the total glycerol derivative content present in the composition. In some embodiments, the 3-(heptyloxy)propane-1,2-diol constitutes from about 10 wt. % to about 99 wt. %, from about 30 wt. % to about 99 wt. %, from about 50 wt. % to about 99 wt. %, from about 70 wt. % to about 99 wt. %, from about 90 wt. % to about 99 wt. %, from about 10 wt. % to about 95 wt. %, from about 30 wt. % to about 95 wt. %, from about 50 wt. % to about 95 wt. %, from about 70 wt. % to about 95 wt. %, from about 90 wt. % to about 95 wt. %, from about 10 wt. % to about 90 wt. %, from about 30 wt. % to about 90 wt. %, from about 50 wt. % to about 90 wt. %, from about 70 wt. % to about 90 wt. %, from about 10 wt. % to about 80 wt. %, from about 30 wt. % to about 80 wt. %, from about 50 wt. % to about 80 wt. %, from about 70 wt. % to about 80 wt. %, from about 10 wt. % to about 70 wt. %, from about 30 wt. % to about 70 wt. %, or from about 50 wt. % to about 70 wt. % of the total glycerol derivative content present in the composition.

The composition can also further comprise one or more C₄ to C₁₀ alkane diols (i.e., in addition to 3-(heptyloxy)propane-1,2-diol), which can also function as preservatives.

In some embodiments, the composition further comprises one or more C₄ to C₁₀ vicinal alkane diols.

For example, the composition can comprise one or more diols at a concentration that is about 1 wt. % or greater, about 2 wt. % or greater, about 5 wt. % or greater, about 10 wt. % or greater, about 15 wt. % or greater, or about 20 wt. % or greater. In some embodiments, the concentration of the one or more diols can be from about 1 wt. % to about 50 wt. %, from about 2 wt. % to about 50 wt. %, from about 5 wt. % to about 50 wt. %, from about 10 wt. % to about 50 wt. %, from about 20 wt. % to about 50 wt. %, from about 1 wt. % to about 25 wt. %, from about 2 wt. % to about 25 wt. %, from about 5 wt. % to about 25 wt. %, from about 10 wt. % to about 25 wt. %, from about 1 wt. % to about 15 wt. %, from about 2 wt. % to about 15 wt. %, from about 5 wt. % to about 15 wt. %, from about 10 wt. % to about 15 wt. %, from about 1 wt. % to about 10 wt. %, from about 2 wt. % to about 10 wt. %, from about 5 wt. % to about 10 wt. %, or from about 1 wt. % to about 5 wt. %.

In various embodiments, the weight ratio of 3-(heptyloxy)propane-1,2-diol to the one or more diols can be from about 1:20 to about 20:1, from about 1:10 to about 10:1, from about 5:1 to about 1:5, or about 1:1. Further, the amount of 3-(heptyloxy)propane-1,2-diol can be in excess relative to the one or more diols. For example, the weight ratio of 3-(heptyloxy)propane-1,2-diol to the one or more diols can be about 1.1:1 or greater, about 1.5:1 or greater, about 2:1 or greater, about 3:1 or greater, about 4:1 or greater, about 5:1 or greater, about 10:1 or greater, or about 25:1 or greater. In some embodiments, the weight ratio of 3-(heptyloxy)propane-1,2-diol to the one or more diols is from about 1:1 to about 100:1, from about 1:1 to about 50:1, from about 1:1 to about 20:1, from about 1.1:1 to about 100:1, from about 1.1:1 to about 50:1, from about 1.1:1 to about 20:1, from about 2:1 to about 100:1, from about 2:1 to about 50:1, from about 2:1 to about 20:1, from about 5:1 to about 100:1, from about 5:1 to about 50:1, from about 5:1 to about 20:1, from about 10:1 to about 100:1, from about 10:1 to about 50:1, from about 10:1 to about 20:1, from about 20:1 to about 100:1, from about 20:1 to about 50:1, or from about 50:1 to about 100:1.

In some embodiments, the 3-(heptyloxy)propane-1,2-diol constitutes about 10 wt. % or greater, about 20 wt. % or greater, about 30 wt. % or greater, about 40 wt. % or greater, about 50 wt. % or greater, about 60 wt. % or greater, about 70 wt. % or greater, about 80 wt. % or greater, about 90 wt. % or greater, or about 95 wt. % or greater of the total diol content present in the composition. In some embodiments, the 3-(heptyloxy)propane-1,2-diol constitutes from about 10 wt. % to about 99 wt. %, from about 30 wt. % to about 99 wt. %, from about 50 wt. % to about 99 wt. %, from about 70 wt. % to about 99 wt. %, from about 90 wt. % to about 99 wt. %, from about 10 wt. % to about 95 wt. %, from about 30 wt. % to about 95 wt. %, from about 50 wt. % to about 95 wt. %, from about 70 wt. % to about 95 wt. %, from about 90 wt. % to about 95 wt. %, from about 10 wt. % to about 90 wt. %, from about 30 wt. % to about 90 wt. %, from about 50 wt. % to about 90 wt. %, from about 70 wt. % to about 90 wt. %, from about 10 wt. % to about 80 wt. %, from about 30 wt. % to about 80 wt. %, from about 50 wt. % to about 80 wt. %, from about 70 wt. % to about 80 wt. %, from about 10 wt. % to about 70 wt. %, from about 30 wt. % to about 70 wt. %, or from about 50 wt. % to about 70 wt. % of the total diol content present in the composition.

In various embodiments, the additional ingredient (or additional preservative) comprises at least one glycerol derivative selected from the group consisting of 3-[(2-ethylhexyl)oxy]-1,2-propanediol, 3-[(n-octyl)oxy]-1,2-propanediol, and 3-[(2-octyl)oxy]-1,2-propanediol. In some embodiments, the additional ingredient comprises at least one glycerol derivative selected from the group consisting of butyl glycerylether, (iso)amylglycerylether, hexylglycerylether, 3-octylglycerylether, 1-nonylglycerylether, and 1-dodecylglycerylether.

As noted, the composition can contain one or more additional preservatives in combination with 3-(heptyloxy)propane-1,2-diol. For example, the preservative can further comprise a propanediol such as 1,3-propandiol, 3-[(2-ethylhexyl)oxy]-1,2-propanediol, 3-[(n-octyl)oxy]-1,2-propanediol, 3-[(2-octyl)oxy]-1,2-propanediol, methyl propanediol, hexoxy-propan-1,2-diol, heptoxy-propan-1,2-diol, octoxy-propan-1,2-diol, 3-phenoxy-propan-1,2-diol, 3-benzyloxy-propan-1,2-diol, 3-phenylethyloxy-propan-1,2-diol, 3-phenylpropyloxy-propan-1,2-diol, 3-methylbenzyloxy-propan-1,2-diol, 2-bromo-2-nitro-1,3-propanediol, 3-(4-chlorphenoxy)-1,2-propanediol (chlorphenesin), or 3-(4-chlorophenoxy)-1,2-propanediol. In some embodiments, the preservative further comprises a pentanediol such as 1,5-pentanediol and 1,2-pentanediol. In certain embodiments, the preservative further comprises a butanediol such as 2,3-butanediol. In other embodiments, the preservative further comprises a hexanediol such as 1,2-hexanediol and 1,6-hexanediol. In various embodiments, the preservative further comprises an octanediol such as 1,2-octanediol and 1,8-octanediol. In still other embodiments, the preservative further comprises a decanediol such as 1,2-decanediol and 1,10-decanediol.

Other examples of additional preservatives that can be used in combination with 3-(heptyloxy)propane-1,2-diol include thymol, eugenol, benzyl alcohol, 2-phenylethyl alcohol, 3-phenyl propanol, 2-phenoxyethanol, 1-phenoxy-propan-2-ol, 3-phenoxypropanol, benzyloxymethanol, phenoxyisopropanol, phenethyl alcohol, phenylpropanol, cinnamic alcohol, 2,4-dichlorobenzyl alcohol, (ethylendioxy)dimethanol, α,α′,α″-trimethyl-1,3,5-triazine-1,3,5(2H,4H,6H)-triethanol, (1-hydroxy-4-methyl-6-(2,4,4-trimethylpentyl)-2(1H)-pyridone, 2-aminoethanol), farnesol, 2-benzylheptan-1-ol, pentylene glycol, caprylyl glycol, chloroxylenol, phenoxyethanol, 4-chloro-m-cresol, aryloxy alcohols [phenoxypropanols (such as 1-phenoxypropan-2-ol, 2-phenoxypropan-1-ol, and 3-phenoxypropan-1-ol)], oligoalkanol aryl ethers [phenoxydiethanol, phenoxytriethanol, phenoxyoligoethanol, phenoxydipropanol, phenoxytripropanol, and phenoxyoligopropanol], aryl alkanols [3-phenylpropan-1-ol, veratryl alcohol (3,4-dimethoxyphenlmethyl alcohol), and 2-methyl-1-phenyl-2-propanol]. In further embodiments, the additional preservative is selected from the group consisting of 4-chloro-3,5-dimethylphenol, bromochlorophene, 2,2′-methylenebis(6-bromo-4-chlorophenol), 3-methyl-4-(1-methylethyl)phenol, 2-benzyl-4-chloro-phenol, and combinations thereof.

The additional preservative can also be an acetophenone derivative such as hydroxyacetophenone, 2-hydroxyacetophenone, 3-hydroxyacetophenone, 4-hydroxyacetophenone, or combinations thereof.

Further, the additional preservative can be selected from the group consisting of tetrahydro-1,3,4,6-tetrakis(hydroxymethyl)imidazo[4,5-d]imidazol-2,5(1H,3H)-dion, 1,3-bis(hydroxymethyl)-5,5-dimethylimidazolidine-2,4-dione, 1,3-bis-(hydroxymethyl)-5,5-dimethyl-2,4-imidazolidinedione, 1,6-bis(4-amidino-2-bromophenoxy)-n-hexane and its salts, 1,6-bis(4-amidino-phenoxy)-n-hexane and its salts, chlorhexidine, 5-bromo-5-nitro-1,3-dioxane, 5-ethyl-1-aza-3,7-dioxabicyclo(3.3.0)octane, 1,2-dibromo-2,4-dicyanobutane, 1-(4-chlorophenoxy)-1-(1H-imidazol-1-yl)-3,3-dimethyl-2-butanone, and combinations thereof.

Other examples of additional preservatives include 2,4,4′-trichloro-2′-hydroxy-diphenyl ether (triclosan), 2-hydroxybiphenyl ether and its salts, and combinations thereof as well as aldehydes such as formaldehyde and paraformaldehyde, 4-hydroxy benzaldehyde, ortho-, meta-, and para-anisic aldehyde, cinnamic aldehyde, or glutaraldehyde.

The additional preservative can also be benzoic acid, para-hydroxybenzoic acid, paraoxybenzoic ester, benzyl benzoate, sodium benzoate, propionic acid, salicylic acid, salicylic acid N-alkylamides, such as, for example, n-octylsalicylamide or n-decylsalicylamide, 2,4-hexadienoic acid (sorbic acid), hydroxyethylglycine of sorbic acid, levulinic acid, anisic acid, perillic acid, cinnamic acid, dehydroacetic acid, formic acid, 10-undecylenic acid, caprylhydroxamic acid, sorbohydroxamic acid, 4-hydroxybenzoic acid, proprionic acid, lactic acid, 2-butyloctanoic acid, and various salts and esters thereof.

The additional preservative can also include one or more of N-(4-chlorophenyl)-N′-(3,4-dichlorophenyl)-urea, 1,1′-methylene-bis(3-(1-hydroxymethyl-2,4-dioximidazolidin-5-yl)urea), N-hydroxymethyl-N-(1,3-di(hydroxymethyl)-2,5-dioxoimidazolidin-4-yl)-N′-hydroxy-methylurea, imidazolidinylurea, 1,3-bis(hydroxymethyl)-1-(1,3,4-tris(hydroxymethyl)-2,5-dioxoimidazolidin-4-yl)urea (diazolidinyl urea), and imidazolidinyl urea.

The additional preservative can include one or more fatty acid esters such as monoglyceride, polyglyceride, medium-chain triglyceride, propanediol caprylate, fatty acid ester of a monohydric alcohol, octadecanoic monoglycerylester (derived from octadecanoic acid), octadecanoic acid monoester with 1,2-propanediol, octadecanoic acid monoester with glycerol, undec-10-enoic acid monoester with a diol, undec-10-enoic acid monoester with propane-1,3-diol, and undec-10-enoic acid monoester with glycerol.

The additional preservative can be a paraben or its salt such as benzyl paraben, butyl paraben, ethyl paraben, isobutyl paraben, isopropyl paraben, methyl paraben, propyl paraben, and combinations thereof.

Still further examples of additional preservatives include heavy metal citrate salts, piroctose, zinc salts, pyrithione and its heavy metal salts, zinc phenol sulfate, zinc undecylenate, bis(2-pyridylthio)zinc 1,1′-dioxide, zinc pyrithione, 2-zinc-sulfidopyridine N-oxide, and combinations thereof.

The additional preservative can also include one or more anti-fungal agents selected from the group consisting of ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, (RS)-1-(4-chlorophenoxy)-1-imidazol-1-yl-3,3-dimethylbutan-2-one (climbazole), and combinations thereof.

Other additional preservatives include N,N′-(decane-1,10-diyldipyridin-1-yl-4-ylidene)-dioctan-1-amine dihydrochloride (octenidine dihydrochloride) (octenidine), cetyltrimethyl ammonium chloride, cetylpyridinium chloride, benzethonium chloride, diisobutylethoxyethyldimethyl benzylammonium chloride, tetramethylammonium chloride, tricetylmethylammonium chloride, silver chloride (AgCl), benzalkonium chloride, poly-(hexamethylenediguanide) hydrochloride, 1-(3-chloroallyl)-3,5,7-triaza-1-azonia-adamantane chloride, N-alkyl(C₁₂-C₂₂)trimethyl-ammonium chloride, alkyl-(C₈-C₁₈)-dimethyl-benzyl-ammonium chloride, cetyl pyridium chloride, chlorhexidine hydrochloride, and combinations thereof. In another embodiments, the preservative is N-alkyl(C₁₂-C₂₂)trimethyl-ammonium bromide and/or alkyl-(C₈-C₁₈)-dimethyl-benzylammonium bromide.

Further examples of additional preservatives comprise diaminoalkylamide, L-lysine hexadecylamide, 2-chloroacetamide, hyamines, (benzyloxymethoxy)-methanol hexamethylenetetramine, selenium disulfide, thiamine dilauryl sulfite, metabisulfite, thiamine dilauryl sulphate (TDS), sodium aluminium chlorohydroxylactate, chlorhexidine acetate, sodium hydroxymethyl-aminoacetate, sodium N-lauryl sarcosinate, sodium-N-palmethyl sarcosinate, lauroyl sarcosine, potassium-N-laurylsarcosine, sodium iodate, sodium salts of diethylhexylsulfosuccinate, ascorbyl palmitate, ethyl lauroyl arginate HCl, glycerol monolaurate (GML), glyceryl caprylate, glycerol monocaprate, glycerol monocaprylate, sorbitan caprylate, diglycerol monocaprate (DMC), sodium anisate, sodium lauroyl lactylate, xylityl sesquicaprylate, triethylcitrate, polyaminopropyl biguanide, chlorhexidine gluconate, alkyl-(C₈-C₁₈)-dimethyl-benzylammonium saccharinate, 3-iodo-2-propynyl butylcarbamate, iodopropynyl butylcarbamate, methyldibromoglutaronitrile, sodium hydroxymethyl-aminoacetate, sodium hydroxymethylglycinate, and combinations thereof.

The additional preservative can also include one or more of 5-chloro-2-methyl-3(2H)-isothiazolinone, 2-methyl-3(2H)-isothiazolinone, methylchloroisothiazolinone, methylisothiazolinone, 2-octyl-2H-isothiazol-3-one, 1,2-benzisothiazol-3(2H)-one, 3-Acetyl-2-hydroxy-6-methyl-4H-pyran-4-one, chlorobutanolum, 5-amino-1,3-bis(2-ethylhexyl)-5-methyl-hexahydropyrimidine, 4,4-dimethyl-1,3-oxazolidine, benzyl hemiformal, Tropolone, 2-Brom-2-(brommethyl)pentandinitril, N-(3-Aminopropyl)-N-dodecylpropan-1,3-diamin, pyridine-2-thiol-1-oxide, naftifine, terbinafine, octopirox (piroctone olamine), chitosan, polyepsilon-lysine, N-myristoylglycine, dimethylol dimethyl hydantoin (DMDMH), DMDM hydantoin, and 3,4,4′-trichlorocarbanilide (TTC or triclocarban).

The additional preservative can further include one or more of Lactobacillus ferment lysate, Lactobacillus/radish root ferment filtrate, thyme oil, oil of cloves, menthol, and mint oil.

In various embodiments, the additional preservative(s) comprises a natural preservative (e.g., obtained or derived from natural sources). In some embodiments, the additional preservatives comprise at least one natural preservatives according to COSMOS standards. For example, the natural preservative can include one or more of 1,3-propandiol; 1,5-pentanediol; 2,3-butanediol; ascorbyl palmitate; ethyl lauroyl arginate HCl; glyceryl caprylate; hydroxyacetophenone; Lactobacillus ferment lysate; Lactobacillus/radish root ferment filtrate; pentylene glycol; phenethyl alcohol; phenylpropanol; polyepsilon-lysine; sodium anisate; sodium lauroyl lactylate; xylityl sesquicaprylate; zinc undecylenate, phenoxyethanol, and glucono-delta-lactone.

In various embodiments, the additional ingredient (or additional preservative) comprises (iso-)amylglycerylether.

In various embodiments, the additional ingredient (or additional preservative) comprises hexanediol.

In various embodiments, the additional ingredient comprises a betaine (e.g., cocamidopropyl betaine).

In various embodiments, the additional ingredient (or additional preservative) comprises hydroxyacetophenone (HAP).

In various embodiments, the additional ingredient (or additional preservative) comprises 3-[(n-octyl)oxy]-1,2-propanediol.

In various embodiments, the additional ingredient (or additional preservative) comprises 3-[(2-octyl)oxy]-1,2-propanediol.

In various embodiments, the additional ingredient (or additional preservative) comprises 2-ethylhexyl glycerylether.

Various combinations of preservatives (e.g., any combination of preservatives noted above) can be used.

### Additional Ingredients—Rheology Modifiers

The compositions described herein can also further comprise a rheology modifier (i.e., a thickener) to increase the viscosity of the composition. Examples of rheology modifiers include hydrophilic silicas, polysaccharides, xanthan gum, dehydroxanthan gum, guar gum, karaya gum, agar-agar, alginates, carrageenan, carob seed flour, hydrolyzed corn starch, tyloses, carboxymethyl cellulose, hydroxyethyl cellulose, various cationic starches, guars (e.g., guar hydroxypropyltrimonium chloride), carbomers, high molecular weight polyethylene glycol monoesters and diesters of fatty acids, polyacrylates, polyacrylamides, polyvinyl alcohol, polyvinyl pyrrolidone, ethoxylated fatty acid glycerides, esters of fatty acids with polyols such as pentaerythritol or trimethylol propane, narrow-range fatty alcohol ethoxylates, laureth-2, -3, and -4, and salts such as sodium chloride and ammonium chloride. In certain embodiments, the rheology modifier is a polymer.

In various embodiments, the rheology modifier comprises an inorganic salt. The inorganic salt can be sodium chloride, ammonium chloride, or mixtures thereof. In various embodiments, the concentration of inorganic salt ranges from about 0.01 wt. % to about 5 wt. %, from about 0.01 wt. % to about 3 wt. %, from about 0.1 wt. % to about 3 wt. %, or about 0.1 wt. % to about 1 wt. %. In various embodiments, the concentration of inorganic salt is less than about 1 wt. %, for example, 1 wt. %, 0.9 wt. %, 0.8 wt. %, 0.7 wt. %, 0.6 wt. %, 0.5 wt. %, 0.4 wt. %, 0.3 wt. %, 0.2 wt. %, 0.1 wt. %, or 0.01 wt. %. In various embodiments, the concentration of inorganic salt is 0% wt. % (i.e., the composition does not comprise any inorganic salt). In various embodiments, the composition is essentially free of rheology modifiers. In various embodiments, the composition is free of a rheology modifier.

As noted, it has been discovered that 3-(heptyloxy)propane-1,2-diol is a multi-functional ingredient. For example, this ingredient can function as a thickener/rheology modifier. Accordingly, in various embodiments, the composition exhibits enhanced rheology characteristics without use of an additional rheology modifier. Also, 3-(heptyloxy)propane-1,2-diol provides for compositions requiring fewer additives as compared to existing compositions. As such, in some embodiments, the composition is free or essentially free (e.g., <1 wt. %, <0.1 wt. %, or even <0.01 wt. % of the total composition) of an additional rheology modifier. For example, the composition can be free or essentially free (e.g., <1 wt. %, <0.1 wt. %, or even <0.01 wt. % of the total composition) of one or more additional rheology modifiers (e.g., free or essentially free of one or more rheology modifiers listed above). In the case of inorganic salt rheology modifiers, a reduction in concentration or absence of inorganic salt provides a cosmetic composition resulting in reduced dryness, irritation, and damage to hair or skin when applied.

### Additional Ingredients—Surfactants

The compositions described herein can also further comprise at least one surfactant. Surfactants generally include various anionic surfactants, cationic surfactants, non-ionic surfactants, zwitterionic surfactants and/or amphoteric surfactants. In some embodiments, the composition contains one or more anionic surfactants, cationic surfactants, non-ionic surfactants, zwitterionic surfactants and/or amphoteric surfactants that are cosmetically and/or pharmaceutically acceptable. In some embodiments, the at least one surfactant comprises an anionic surfactant, a non-ionic surfactant, an amphoteric surfactant, or mixtures thereof.

In some embodiments, the at least one surfactant comprises an anionic surfactant, wherein the anionic surfactant comprises a sulfated surfactant. In some embodiments, the at least one surfactant comprises a mixture of at least one anionic surfactant and at least one non-ionic surfactant or a mixture of at least one anionic surfactant and at least one amphoteric surfactant. In some embodiments, the anionic surfactant comprises a sulfated surfactant, the non-ionic surfactant comprises an alkylpolyglucoside, and/or the amphoteric surfactant comprises a betaine.

In some embodiments, the composition has a surfactant concentration of about 1 wt. % or greater, about 2 wt. % or greater, about 5 wt. % or greater, about 10 wt. % or greater, about 20 wt. % or greater, about 30 wt. % or greater, about 40 wt. % or greater, or about 50 wt. % or greater. In certain embodiments, the composition has a surfactant concentration of from about 1 wt. % to about 60 wt. %, from about 2 wt. % to about 60 wt. %, from about 5 wt. % to about 60 wt. %, from about 10 wt. % to about 60 wt. %, from about 20 wt. % to about 60 wt. %, from about 1 wt. % to about 50 wt. %, from about 2 wt. % to about 50 wt. %, from about 5 wt. % to about 50 wt. %, from about 10 wt. % to about 50 wt. %, from about 20 wt. % to about 50 wt. %, from about 1 wt. % to about 25 wt. %, from about 2 wt. % to about 25 wt. %, from about 5 wt. % to about 25 wt. %, from about 10 wt. % to about 25 wt. %, from about 1 wt. % to about 15 wt. %, from about 2 wt. % to about 15 wt. %, from about 5 wt. % to about 15 wt. %, from about 10 wt. % to about 15 wt. %, from about 1 wt. % to about 10 wt. %, from about 2 wt. % to about 10 wt. %, from about 5 wt. % to about 10 wt. %, or from about 1 wt. % to about 5 wt. %.

Examples anionic surfactants include for example, alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, a-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, other sulfated surfactants, alkylglyceride ether sulfonates, fatty acid methyltaurides, fatty acid sarcosinates, sulforicinoleates, acylglutamates, mixtures thereof, and salts thereof.

A type of anionic surfactants is sulfated surfactants. Examples of sulfated surfactants include sodium cocoyl sulfate, sodium lauryl sulfate, ammonium lauryl sulfate, sodium laureth sulfate, ammonium coco-sulfate, magnesium lauryl sulfate, magnesium laureth sulfate, ammonium laureth sulfate, sodium cetearyl sulfate, sodium myreth sulfate, magnesium myreth sulfate, ammonium myreth sulfate, and sodium C₁₂ to C₁₈ alkyl sulfate.

In various embodiments, the composition comprises one or more non-ionic surfactants. Examples of non-ionic surfactants include various ethoxylated surfactants, such as alkyl ethoxylates, ethoxylated fatty alcohols, ethoxylated fatty acids, ethoxylated fatty acid glycerides or ethoxylated alkylphenols; mono- or di-alkyl alkanolamides; and alkylpolysaccharides.

In certain embodiments, the composition can comprise one or more non-ionic surfactants selected from the group consisting of fatty alcohol ethoxylates (alkylpolyethylene glycols), alkylphenol polyethylene glycols, alkylmercaptan polyethylene glycols, fatty amine ethoxylates (alkylaminopolyethylene glycols), fatty acid ethoxylates (acylpolyethylene glycols), polypropylene glycol ethoxylates, fatty acid alkylol amides, (fatty acid amide polyethylene glycols), N-alkyl-, N-alkoxypolyhydroxy-fatty acid amides, sucrose esters, sorbitol esters, polyglycol ethers, and mixtures thereof. Other sugar-derived nonionic surfactants which can be included in compositions of the invention include the fatty N-alkyl polyhydroxy fatty acid amides.

In some embodiments, the composition comprises one or more alkylpolysaccharide surfactants. Examples of alkylpolysaccharide surfactants include compounds of the following formula:

R—O-(sug)ᵤ

wherein R is a straight or branched chain substituted or unsubstituted hydrocarbyl selected from alkyl, alkenyl, alkylphenyl, alkenylphenyl having from about 4 to about 30 carbon atoms or from about 4 to 20 carbon atoms. The sug moiety is a saccharide residue, and may be an open or cyclic (i.e., pyranose) structure. The saccharide may be a monosaccharide having 5 or 6 carbon atoms, a disaccharide, an oligosaccharide or a polysaccharide. Examples of suitable saccharide moieties, including their corresponding pyranose form, include ribose, xylose, arabinose, glucose, galactose, mannose, telose, gulose, allose, altrose, idose, lyxose, ribulose, sorbose (sorbitan), fructose, and mixtures thereof. Examples of suitable disaccharides include maltose, lactose, and sucrose. Disaccharides, oligosaccharides, and polysaccharides can be a combination of two or more identical saccharides, for example maltose (two glucoses) or two or more different saccharides, for example sucrose (a combination of glucose and fructose). The degree of polymerization, u, is an average number from 1 to about 50, from 1 to about 25, from 1 to about 10, from 1 to about 5, from 1 to about 3, or from 1 to about 2.

In various embodiments, the alkylpolysaccharide surfactant can be an alkylpolyglucoside (APG) surfactant. For example, some APGs are of the formula above wherein: R is a branched or straight chain alkyl group preferably having from 4 to 30 carbon atoms or from 4 to 20 carbon atoms, or a mixture of alkyl groups having an average value within the given range; sug is a glucose residue (e.g., a glycoside); and u is from 1 to about 5, and more preferably from 1 to about 3. In various embodiments, the surfactant comprises one or more APGs of the formula above wherein R is a branched or straight chain alkyl group having from 5 to 20 carbon atoms or 6 to 16 carbon atoms or a mixture of alkyl groups having an average value within the given range and u is from 1 to about 3. The alkyl polyglucoside can be selected from the group consisting of caprylyl/capcyl glucoside, coco glucoside, and sucrose glucoside.

In certain embodiments, the composition comprises one or more amphoteric surfactants selected from the group consisting of N-alkyl-p-aminopropionates and N-alkyl-p-iminodipropionates as alkali metal salts and mono-, di-, and trialkylammonium salts; N-acylaminoalkyl-N,N-dimethylacetobetaine (e.g., N-acylaminopropyl-N,N-dimethylacetobetaine, alkyl-dimethylsulfopropylbetaine, amphosurfactants based on imidazoline, (e.g., sodium salt of 1-(P-carboxymethyloxyethyl)-1-(carboxymethyl)-2-laurylimidazolinium; amine oxides, (e.g., alkyl-dimethylamine oxide), and fatty acid amidoalkyldimethylamine oxide.

In certain embodiments, the composition comprises one or more betaine surfactants. For example, the betaine surfactant can be selected from alkylbetaines (e.g., cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine); sulfobetaines (e.g., cocodimethylsulfopropylbetaine, stearyldimethylsulfopropylbetaine, lauryldimethyl-sulfoethylbetaine, and laurylbis(2-hydroxyethyl)sulfopropylbetaine); betaines that are carboxyl derivatives of imidazole; betaines that are alkyldimethylammonium acetates; betaines that alkyldimethylcarbonylmethylammonium salts; and fatty acid alkylamidobetaines (e.g., coconut fatty acid amidopropylbetaine, and N-coconut fatty acid amidoethyl-N-[2-(carboxymethoxy)ethyl]glycerol). One preferred betaine surfactant is cocamidopropyl betaine.

In various embodiments, composition comprises at least one surfactant selected from the group consisting of an alkylpolyglucoside, sodium cocosulfate, sodium lauryl sulfate, ammonium lauryl sulfate, sodium lauryl ether sulfate, sodium cocoylglutamate, sodium cocoylglycinate, sodium lauroyl sarcosinate, sodium lauryl sulfoacetate, lauryl glucose carboxylate, cocamidopropyl betaine, lauryl glucoside, decyl glucoside, caprylyl/capryl glucoside, coco-glucoside, hexyl glucoside, disodium cocoyl glutamate, sodium stearoyl glutamate, sodium palmoyl glutamate, sodium lauroyl glutamate, sodium myristoyl glutamate, potassium cocoyl glutamate, potassium lauroyl glutamate, potassium myristoyl glutamate, sodium lauroyl glycinate, potassium cocoyl glycinate, potassium cocoyl glycinate, sodium cocoyl isethionate, sodium cocoyl methyl isethionate, sodium lauroyl methyl isethionate, sodium methyl oleoyl taurate, sodium methyl lauroyl taurate, sodium methyl cocoyl taurate, cocoyl methyl glucamide, capryloyl/caproyl methyl glucamide, lauroyl/myristoyl methyl glucamide, cocobetaine, cocamidopropyl hydroxysultaine, lauryl betaine, sodium cocoamphoacetate, disodium cocoamphodiacetate, sodium lauroamphoacetate, sodium lauryl sulfoacetate, disodium laureth sulfosuccinate, disodium oleamido MIPA sulfosuccinate, cocamide DEA, cocamide MEA, cocamide MIPA, Sodium C₁₄₋₁₆ olefin sulfonate, potassium cocoate, potassium palmate, potassium laurate, potassium olivate, sodium cocoyl sulfate, sodium lauryl sulfate, ammonium lauryl sulfate, sodium laureth sulfate, ammonium coco-sulfate, magnesium lauryl sulfate, magnesium laureth sulfate, ammonium laureth sulfate, sodium cetearyl sulfate, sodium myreth sulfate, magnesium myreth sulfate, ammonium myreth sulfate, sodium C₁₂ to C₁₈ alkyl sulfate, and mixtures thereof.

In some embodiments, the total concentration of surfactants in the composition can be from about 1% wt. % to about 30% wt. %, from about 10% wt. % to about 25% wt. %, or from about to 12% wt. % to about 24% wt. %. In some embodiments, the concentration of surfactant in the composition is about 12% wt. %, 21% wt. %, or 24% wt. %.

In some embodiments, the concentration of the sulfated surfactant in the composition can be from about 1% wt. % to about 15% wt. %, from about 5% wt. % to about 12% wt. %, or from about 8% wt. % to about 10% wt. %. In some embodiments, the concentration of the sulfated surfactant is about 8% wt. % or 10% wt. %.

### Additional Ingredients—Antioxidants

In some embodiments, the composition further comprises an antioxidant. In various embodiments, the antioxidant(s) comprises a natural antioxidant (e.g., obtained or derived from natural sources). In some embodiments, the antioxidant comprises a natural antioxidant according to COSMOS standards. For example, the antioxidant can include one or more of 5,7-dihydroxy-2-(4-hydroxyphenyl)chroman-4-one (naringenin); 2-(3,4-dihydroxyphenyl)-5,7-dihydroxy-4H-1-benzopyran-4-one (quercetin); 5,7-dihydroxy-2-(3-hydroxy-4-methoxyphenyl)chroman-4-one (hesperetin); 5,7-dihydroxy-2-(3,4-dihydroxyphenyl)-chroman-4-one (eriodyctiol), and combinations thereof.

Various examples of antioxidants include (i) flavones such as taxifolin (epicatechin), isokuranetin, aromadendrin (aromadedrin), acacetin, isocutellarein, luteolin, kaempferol, apigenin, diosmetin, chrysoeriol, chrysin, galangin, limocitrin; flavonols such as azaleatin, fisetin, gossypetin, kaempferide, isorhamnetin, morin, myricetin, natsudaidain, pachypodol, rhamnazin, and rhamnetin; (ii) flavonones such as eriocitrin, narirutin, hesperidin, neoponcirin, neoeriocitrin, naringenin, neohesperiden, poncirin, isorhoifolin, deosmin, rhoifolin, and neodeosmin; (iii) glycosides formed from a flavonol such as astragalin, azalein, hyperoside, isoquercitin, kaempferitrin, myricitrin, quercitrin, robinin, rutin, spiraeoside, and xanthorhamnin; (iv) polymethoxylated flavones such as sinensetin, nobiletin, heptamethoxyflavone, natsudaidain, 5-demethylnobiletin, tangeretin, tetra-O-methylscutellerein, tetra-O-methylisoscutellarein, hexo-O-methylquercetagetin, hexa-O-methylgossypetin, and 5-hydroxy-3,7,8,3′,4′-pentamethoxyflavone; and (v) flavonoid or glycosylated precursors thereof, such as derivatives of quercetin, preferably alpha-glucosyl rutin, carnosol, carnosolic acid, resveratrol, sinapic acid, curcuminoids, retinoids, (e.g., retinyl palmitate, retinol or tretinoin), ursolic acid, levulinic acid, butyl hydroxytoluene, butyl hydroxyanisole, nordihydroguaiac acid, trihydroxybutyrophenone, and uric acid.

The antioxidant can also include one or more of amurensin, apigetrin, baicalein, butin, diosmin, eriodictyol, eupafolin, eupatilin, flavoxate, genkwanin, hispidulin, homoeriodictyol, icariin, isosakuranetin, likvirtin, liquiritin, liquiritigenin, neohesperidin, pinocembrin, robinose, rutinose, sakuranetin, sakuranin, scutellarein, spirenoside, sterubin, tangeritin, techtochrysin, troxerutin, and wogonin.

The antioxidant can also include an imidazole (e.g., urocanic acid) and derivatives thereof.

Other antioxidants include one or more amino acids such as glycine, histidine, tyrosine, tryptophan, and cysteine or a derivative thereof such as cysteine hydrochloride, acetylcysteine, and N-acylamino acids and salts thereof (e.g. N-octanoylglycine, Lipacide C8G); and/or a peptides, such as D,L-carnosine, D-camosine, L-carnosine and derivatives thereof (preferably anserine), carnitine, creatine, matrikine peptides (e.g., lysyl-threonyl-threonyl-lysyl-serine) and palmitoylated pentapeptides.

The antioxidant can include one or more carotenoids, carotenes (preferably alpha-carotene, beta-carotene, lycopene), and derivatives thereof.

Other antioxidants include lipoic acid and derivatives thereof (e.g., dihydrolipoic acid), erythorbic acid, ethyl ferulate, ferulic acid, kojic acid, nordihydroguaiaretic acid, phenylthioglycolic acid, thiodiglycolic acid, thioglycolic acid, thiolactic acid, thiosalicylic acid, alpha-hydroxycarboxylic acids (e.g., glycolic acid, mandelic acid) and salts thereof, p-hydroxybenzoic esters (e.g., methyl, ethyl, propyl, or butyl esters thereof), rosmarinic acid, coniferyl benzoate of benzoic resin, rutinic acid and derivatives thereof, folic acid and derivatives thereof, ubiquinone and derivatives thereof, ubiquinol and derivatives thereof, vitamin C and derivatives (preferably ascorbyl palmitate, Mg ascorbyl phosphate, ascorbyl acetate, ascorbyl glucoside), alpha-hydroxy acids (preferably citric acid, malic acid), humic acid, bile acid, bile extracts, tannins, bilirubin, biliverdin, EDTA, EGTA, and derivatives thereof), thiodipropionic acid and derivatives thereof (preferably esters, ethers, peptides, lipids, nucleotides, nucleosides and salts), hydroxy acids, urocaninic acid, phytinic acid, lactic acid, linolenic acid, caffeic acid, and chlorogenic acid.

Fatty acids such as unsaturated fatty acids and derivatives thereof (preferably gamma-linolenic acid, linoleic acid, oleic acid), fatty acids, and hydroxyfatty acids are also suitable antioxidants.

Still other antioxidants include propyl thiouracil or other thiols (e.g., thioredoxine, glutathione, cystine, cystamine and glycosyl, N-acetyl, methyl, ethyl, propyl, amyl, butyl and lauryl, palmitoyl, oleyl, gamma-linoleyl, cholesteryl, glyceryl and oligoglyceryl esters thereof) and salts thereof, thiol glycosyl esters, thiol N-acetyl esters, thiol methyl esters, thiol ethyl esters, thiol propyl esters, thiol amyl esters, thiol butyl esters, thiol lauryl esters, thiol palmitoyl esters, thiol oleyl esters, thiol linoleyl esters, thiol cholesteryl esters, and thiol glyceryl esters.

Antioxidants also include vitamin A and its derivatives such as vitamin A palmitate; and tocophereth-5, tocophereth-10, tocophereth-12, tocophereth-18, tocophereth-50, tocophersolan, tocopherol (e.g., vitamin E), and its derivatives (e.g., vitamin E derivatives such as vitamin E acetate, vitamin E linoleate, vitamin E nicotinate, and vitamin E succinate).

The antioxidant can also include metal chelators such as alpha-hydroxy fatty acids, palmitic acid, phytic acid, or lactoferrin.

Other examples of antioxidants include zinc or derivatives thereof (e.g., ZnO, ZnSO4), selenium or derivatives thereof (e.g., selenium methionine), superoxide dismutase, and stilbenes or derivatives thereof (e.g., stilbene oxide, trans-stilbene oxide).

The antioxidant can include one or more sugars such as mannose, aurothioglucose, and/or derivatives thereof.

Various extracts or a fraction of one or more plants can also function as antioxidants. Examples include extracts or fractions of green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John's wort, grape seeds, wheat germ, and Phyllanthus emblica.

The antioxidant can also include a coenzyme, such as coenzyme Q-10, plastoquinone, and menaquinone.

Even further examples of antioxidants include p-hydroxyanisole, 3-tert-butyl-4-hydroxyanisole, 2,6-di-tert-butyl-p-cresol, dioleyl tocopheryl methylsilanol, phloroglucinol, dexpanthenol, hinokitol, thiodiglycol, thiodiglycolamide, hydroquinone, tert-butylhydroquinone, diamylhydroquinone, di-tert-butylhydroquinone, decylmercaptomethylimidazole, distearyl thiodipropionate, ditridecyl thiodipropionate, dicetyl thiodipropionate, dilauryl thiodipropionate, dimyristyl thiodipropionate,digalloyl trioleate, disodium rutinyl disulphate, hydroxylamine sulphate, dodecyl gallate, octyl gallate, propyl gallate, hydroxylamine hydrochloride, isooctyl thioglycolate, madecassicoside, methoxy-PEG-7-rutinyl succinate, sodium erythorbate, sodium thioglycolate, sorbityl furfural, o-tolylbiguanide, tris(nonylphenyl) phosphite, dimethyloldimethylhydantoin, ubichinon, ubichinol, and carnosine.

Various combinations of antioxidants (e.g., any combination of antioxidants noted above) can be used.

### Additional Ingredients—Other

Various compositions as described herein can comprise at least one additional ingredient. For example, the compositions can include at least one active ingredient (e.g., pharmaceutically active ingredient(s), cosmetic active ingredient(s), and/or biologically active ingredient(s)). Pharmaceutically active ingredients (i.e., medicaments) include, for example, antibacterial, antifungal, antiviral, antipruritis, antihistamine, antiseborrhea, anti-odor, antiseptic/wound healing, anti-inflammatory, anticancer, and immunomodulator drugs and so on.

The additional ingredient(s) of the composition can also include one or more inactive substances or excipients.

In some embodiments, the at least one additional ingredient include one or more medicaments, anti-dandruff agents, skin-brightening agents, self-tanning agents, exfoliants, enzymes, anti-acne agents, deodorants and antiperspirants, pediculocides, oily substances, waxes, other preservatives, emulsifiers, co-emulsifiers, solubilizers, cationic polymers, film formers, superfatting agents, refatting agents, foam stabilizers, stabilizers, preservation boosting ingredients, dyes, pigments, opacifiers, abrasives, absorbents, anticaking agents, bulking agents, pearlizing agents, odorants, perfumes or fragrances, carriers, solvents or diluents, propellants, functional acids, viscosity modifiers, thickening and gelling agents, pH adjusting agents, buffering agents, antioxidants, chelants, astringents, sunscreens, sun protection agents, dermal penetration enhancers, UV filters, skin conditioning agents, emollients, humectants, occlusive agents, anti-foaming agents, flavoring agents, electrolytes, oxidizing agents, reducing agents, and combinations thereof.

In various embodiments, the additional ingredient comprises a natural ingredient (e.g., obtained or derived from a natural source). In some embodiments, the composition consists or consists essentially of (i.e., >95 wt. % or even >99 wt. %) natural ingredients. The natural ingredients can be certified according to COSMOS standards.

The compositions described herein can further comprise at least one chelator selected from the group consisting of distarch phosphate, sodium phytate, triethyl citrate, sodium phytate, sodium gluconate, potassium citrate, and calcium citrate.

The compositions described herein can also further comprise a quaternary ammonium and/or phosphonium salt. Quaternary salts are commonly used as conditioning agents and in some cases are used as preservatives.

### Preservatives Enhancing Additional Properties of Compositions

In some embodiments, the invention is directed to cosmetic compositions comprising: 3-(heptyloxy)propane-1,2-diol; 3-[(n-octyl)oxy]-1,2-propanediol; a surfactant; and a rheology modifier comprising an inorganic salt.

As described above, 3-(heptyloxy)propane-1,2-diol and 3-[(n-octyl)oxy]-1,2-propanediol in combination provide an effective preservative for cosmetic and other compositions.

It was surprisingly discovered that the combination of 3-(heptyloxy)propane-1,2-diol and 3-[(n-octyl)oxy]-1,2-propanediol can also synergistically increase viscosity of a composition further comprising a surfactant as described above, such as a surfactant included in a cosmetic composition used to clean hair and/or skin. The synergistic increase in viscosity was most pronounced in surfactant systems comprising an anionic (sulfated) surfactant, which are commonly used in shampoo and other cleaning cosmetic compositions. Further, increased viscosity resulting from the combination of 3-(heptyloxy)propane-1,2-diol and 3-[(n-octyl)oxy]-1,2-propanediol is also observed in compositions formulated with a surfactant system comprising a mixture of surfactants, such as compositions with an anionic (sulfated) surfactant/non-ionic (APG) surfactant system as well as compositions with an anionic (sulfated) surfactant/amphoteric (betaine) surfactant system.

As described above, inorganic salts such as sodium chloride are used in cosmetic compositions as a rheology modifier (i.e., a thickener) to increase the viscosity of the composition. The enhanced viscosity effect of the combination of 3-(heptyloxy)propane-1,2-diol and 3-[(n-octyl)oxy]-1,2-propanediol is observed in compositions further comprising such an inorganic salt as a rheology modifying agent. In particular, the combination allows the formulation of compositions wherein the concentration of the inorganic salt to achieve a given target viscosity is lower than an otherwise identical composition lacking 3-(heptyloxy)propane-1,2-diol and/or 3-[(n-octyl)oxy]-1,2-propanediol. Further, while this effect was observed in composition that also comprised an inorganic salt rheology modifying agent, synergistic enhanced rheology characteristics (i.e., increased viscosity) as a result of the combination of 3-(heptyloxy)propane-1,2-diol and 3-[(n-octyl)oxy]-1,2-propanediol is also provided in compositions without an inorganic salt rheology agent. This discovery allows for the formulation of compositions with lower or no inorganic salt concentrations as described herein that still exhibit the desired rheology characteristics. Lower organic salt concentrations or the absence of inorganic salts is beneficial because inorganic salts in cosmetic compositions can cause dryness and irritation when applied hair or skin.

As recognized by those skilled in the art, the desired target viscosity exhibited by a composition will vary depending on the intended use of the composition. Compositions in accordance with those embodiments of the invention comprising the combination of 3-(heptyloxy)propane-1,2-diol and 3-[(n-octyl)oxy]-1,2-propanediol can be formulated to exhibit a commercially desirable target viscosity either with or without the inclusion of an inorganic salt or other rheology modifying agent.

It was also surprisingly discovered that when formulating compositions comprising the combination of 3-(heptyloxy)propane-1,2-diol and 3-[(n-octyl)oxy]-1,2-propanediol, the solubility of components in the composition is enhanced when 3-(heptyloxy)propane-1,2-diol and 3-[(n-octyl)oxy]-1,2-propanediol are premixed together before mixing with the other components of the composition as compared to a composition when these compounds are added separately and sequentially. 3-[(n-octyl)oxy]-1,2-propanediol has a lower solubility in aqueous systems as compared to 3-(heptyloxy)propane-1,2-diol. Further, 3-(heptyloxy)propane-1,2-diol provides enhanced solubility effects such that clear compositions can be formulated with less risk of precipitate formation.

### Exemplary Compositions

In some embodiments, the invention is directed to cosmetic compositions comprising: 3-(heptyloxy)propane-1,2-diol; 3-[(n-octyl)oxy]-1,2-propanediol; a surfactant; and a rheology modifier comprising an inorganic salt. In some embodiments, the concentration of the inorganic salt to achieve a given viscosity is lower than an otherwise identical composition lacking 3-(heptyloxy)propane-1,2-diol and/or 3-[(n-octyl)oxy]-1,2-propanediol.

In some embodiments, the invention is directed to shampoo compositions comprising 3-(heptyloxy)propane-1,2-diol; 3-[(n-octyl)oxy]-1,2-propanediol; a surfactant selected from the group consisting of sodium cocoyl sulfate, sodium lauryl sulfate, ammonium lauryl sulfate, sodium laureth sulfate, ammonium coco-sulfate, magnesium lauryl sulfate, magnesium laureth sulfate, ammonium laureth sulfate, sodium cetearyl sulfate, sodium myreth sulfate, magnesium myreth sulfate, ammonium myreth sulfate, and sodium C₁₂ to C₁₈ alkyl sulfate; and an optional rheology modifier comprising an inorganic salt; wherein the concentration of the inorganic salt to achieve a given viscosity is lower than an otherwise identical composition lacking 3-(heptyloxy)propane-1,2-diol and/or 3-[(n-octyl)oxy]-1,2-propanediol. In various embodiments, these compositions are free of a rheology modifier.

Exemplary ingredients and ingredient combinations for use in various compositions (e.g., cosmetic and pharmaceutical compositions) are described in the Tables 1, 2A, and 2B below.

TABLE 1
Exemplary Ingredients

| | |
|---|---|
| Ingredient | |
| No. | Ingredient |

Preservatives

| | |
|---|---|
| P1 | 3-(heptyloxy)propane-1,2-diol |
| P2 | 3-[(n-octyl)oxy]-1,2-propanediol |
| P3 | 2-octylglycerylether |
| P4 | 3-[(2-ethylhexyl)oxy]-1,2-propanediol (ethylhexylglycerin) |
| P5 | butylglycerylether |
| P6 | (iso-)amyl glycerylether (=pentyl) |
| P7 | hexylglyceryl ether |
| P8 | 3-octylglycerylether |
| P9 | 1-nonylglycerylether |
| P10 | 1-dodecylglycerylether |
| P11 | alkane diol(s) (C₄-C₁₀) |
| P12 | 1,3-propanediol |
| P13 | 1,5-pentanediol |
| P14 | 2,3-butanediol |
| P15 | ascorbyl palmitate |
| P16 | ethyl lauroyl arginate HCl |
| P17 | glyceryl caprylate |
| P18 | hydroxyacetophenone |
| P19 | Lactobacillus ferment fysate |
| P20 | Lactobacillus/radish root ferment filtrate |
| P21 | pentylene glycol |
| P22 | phenethyl alcohol |
| P23 | phenylpropanol |
| P24 | polyepsilon-lysine |
| P25 | sodium anisate |
| P26 | sodium lauroyl lactylate |
| P27 | xylityl sesquicaprylate |
| P28 | zinc undecylenate |

Antioxidants

| | |
|---|---|
| A1 | 5,7-dihydroxy-2-(4-hydroxyphenyl)chroman-4-one |
| | (naringenin) |
| A2 | 243,4-dihydroxyphenyl)-3,5,7-trihydroxychromen-4-one |
| | (quercetin) |
| A3 | 5,7-dihydroxy-2-(3-hydroxy-4-methoxyphenyl)chroman-4-one |
| | (hesperetin) |
| A4 | 5,7-dihydroxy-2-(3,4-dihydroxyphenyl)-chroman-4-one |
| | (eriodyctiol) |

Chelators

| | |
|---|---|
| C1 | distarch phosphate |
| C2 | sodium phytate |
| C3 | triethyl citrate |
| C4 | sodium gluconate |
| C5 | potassium citrate |
| C6 | calcium citrate |

Other Ingredients

| | |
|---|---|
| Q1 | quaternary ammonium compound(s) |
| S1 | surfactant(s) |

TABLE 2A
Exemplary Combinations of Ingredients

| Composition No. | Ingredient 1 | Ingredient 2 | Ingredient 3 | Ingredient 4 |
|---|---|---|---|---|
| 1 | P1 | — | — | — |
| 2 | P1 | P2 | — | — |
| 3 | P1 | P3 | — | — |
| 4 | P1 | P4 | — | — |
| 5 | P1 | P5 | — | — |
| 6 | P1 | P6 | — | — |
| 7 | P1 | P7 | — | — |
| 8 | P1 | P8 | — | — |
| 9 | P1 | P9 | — | — |
| 10 | P1 | P10 | — | — |
| 11 | P1 | P11 | — | — |
| 12 | P1 | P12, P13, and/or P14 | — | — |
| 13 | P1 | P2 | P11 | — |
| 14 | P1 | P3 | P11 | — |
| 15 | P1 | P4 | P11 | — |
| 16 | P1 | P5 | P11 | — |
| 17 | P1 | P6 | P11 | — |
| 18 | P1 | P7 | P11 | — |
| 19 | P1 | P8 | P11 | — |
| 20 | P1 | P9 | P11 | — |
| 21 | P1 | P10 | P11 | — |
| 22 | P1 | P12, P13, and/or P14 | P11 | — |
| 23 | P1 | P2 | P12, P13, and/or P14 | — |
| 24 | P1 | P3 | P12, P13, and/or P14 | — |
| 25 | P1 | P4 | P12, P13, and/or P14 | — |
| 26 | P1 | P5 | P12, P13, and/or P14 | — |
| 27 | P1 | P6 | P12, P13, and/or P14 | — |
| 28 | P1 | P7 | P12, P13, and/or P14 | — |
| 29 | P1 | P8 | P12, P13, and/or P14 | — |
| 30 | P1 | P9 | P12, P13, and/or P14 | — |
| 31 | P1 | P10 | P12, P13, and/or P14 | — |
| 32 | P1 | one or more of P15-P28 | — | — |
| 33 | P1 | one or more of P15-P28 | P11 | — |
| 34 | P1 | one or more of P15-P28 | P12, P13, and/or P14 | — |
| 35 | P1 | — | — | one or more of A1-A4 |
| 36 | P1 | P2 | — | one or more of A1-A4 |
| 37 | P1 | P3 | — | one or more of A1-A4 |
| 38 | P1 | P4 | — | one or more of A1-A4 |
| 39 | P1 | P5 | — | one or more of A1-A4 |
| 40 | P1 | P6 | — | one or more of A1-A4 |
| 41 | P1 | P7 | — | one or more of A1-A4 |
| 42 | P1 | P8 | — | one or more of A1-A4 |
| 43 | P1 | P9 | — | one or more of A1-A4 |
| 44 | P1 | P10 | — | one or more of A1-A4 |
| 45 | P1 | P11 | — | one or more of A1-A4 |
| 46 | P1 | P2 | P11 | one or more of A1-A4 |
| 47 | P1 | P3 | P11 | one or more of A1-A4 |
| 48 | P1 | P4 | P11 | one or more of A1-A4 |
| 49 | P1 | P5 | P11 | one or more of A1-A4 |
| 50 | P1 | P6 | P11 | one or more of A1-A4 |
| 51 | P1 | P7 | P11 | one or more of A1-A4 |
| 52 | P1 | P8 | P11 | one or more of A1-A4 |
| 53 | P1 | P9 | P11 | one or more of A1-A4 |
| 54 | P1 | P10 | P11 | one or more of A1-A4 |
| 55 | P1 | P12, P13, and/or P14 | — | one or more of A1-A4 |
| 56 | P1 | P2 | P12, P13, and/or P14 | one or more of A1-A4 |
| 57 | P1 | P3 | P12, P13, and/or P14 | one or more of A1-A4 |
| 58 | P1 | P4 | P12, P13, and/or P14 | one or more of A1-A4 |
| 59 | P1 | P5 | P12, P13, and/or P14 | one or more of A1-A4 |
| 60 | P1 | P6 | P12, P13, and/or P14 | one or more of A1-A4 |
| 61 | P1 | P7 | P12, P13, and/or P14 | one or more of A1-A4 |
| 62 | P1 | P8 | P12, P13, and/or P14 | one or more of A1-A4 |
| 63 | P1 | P9 | P12, P13, and/or P14 | one or more of A1-A4 |
| 64 | P1 | P10 | P12, P13, and/or P14 | one or more of A1-A4 |
| 65 | P1 | one or more of P15-P28 | — | one or more of A1-A4 |
| 66 | P1 | one or more of P15-P28 | P11 | one or more of A1-A4 |
| 67 | P1 | one or more of P15-P28 | P12, P13, and/or P14 | one or more of A1-A4 |

TABLE 2B
Exemplary Combinations of Ingredients

| Composition No. | Ingredient 1 | Ingredient 2 | Ingredient 3 | Ingredient 4 | Ingredient 5 |
|---|---|---|---|---|---|
| 68 | P1 | — | — | — | one or more of |
| | | | | | C1-C6, Q1, and/or |
| | | | | | S1 |
| 69 | P1 | P2 | — | — | one or more of |
| | | | | | C1-C6, Q1, and/or |
| | | | | | S1 |
| 70 | P1 | P3 | — | — | one or more of |
| | | | | | C1-C6, Q1, and/or |
| | | | | | S1 |
| 71 | P1 | P4 | — | — | one or more of |
| | | | | | C1-C6, Q1, and/or |
| | | | | | S1 |
| 72 | P1 | P5 | — | — | one or more of |
| | | | | | C1-C6, Q1, and/or |
| | | | | | S1 |
| 73 | P1 | P6 | — | — | one or more of |
| | | | | | C1-C6, Q1, and/or |
| | | | | | S1 |
| 74 | P1 | P7 | — | — | one or more of |
| | | | | | C1-C6, Q1, and/or |
| | | | | | S1 |
| 75 | P1 | P8 | — | — | one or more of |
| | | | | | C1-C6, Q1, and/or |
| | | | | | S1 |
| 76 | P1 | P9 | — | — | one or more of |
| | | | | | C1-C6, Q1, and/or |
| | | | | | S1 |
| 77 | P1 | P10 | — | — | one or more of |
| | | | | | C1-C6, Q1, and/or |
| | | | | | S1 |
| 78 | P1 | P11 | — | — | one or more of |
| | | | | | C1-C6, Q1, and/or |
| | | | | | S1 |
| 79 | P1 | P2 | P11 | — | one or more of |
| | | | | | C1-C6, Q1, and/or |
| | | | | | S1 |
| 80 | P1 | P3 | P11 | — | one or more of |
| | | | | | C1-C6, Q1, and/or |
| | | | | | S1 |
| 81 | P1 | P4 | P11 | — | one or more of |
| | | | | | C1-C6, Q1, and/or |
| | | | | | S1 |
| 82 | P1 | P5 | P11 | — | one or more of |
| | | | | | C1-C6, Q1, and/or |
| | | | | | S1 |
| 83 | P1 | P6 | P11 | — | one or more of |
| | | | | | C1-C6, Q1, and/or |
| | | | | | S1 |
| 84 | P1 | P7 | P11 | — | one or more of |
| | | | | | C1-C6, Q1, and/or |
| | | | | | S1 |
| 85 | P1 | P8 | P11 | — | one or more of |
| | | | | | C1-C6, Q1, and/or |
| | | | | | S1 |
| 86 | P1 | P9 | P11 | — | one or more of |
| | | | | | C1-C6, Q1, and/or |
| | | | | | S1 |
| 87 | P1 | P10 | P11 | — | one or more of |
| | | | | | C1-C6, Q1, and/or |
| | | | | | S1 |
| 88 | P1 | P12, P13, and/or P14 | — | — | one or more of |
| | | | | | C1-C6, Q1, and/or |
| | | | | | S1 |
| 89 | P1 | P2 | P12, P13, and/or P14 | — | one or more of |
| | | | | | C1-C6, Q1, and/or |
| | | | | | S1 |
| 90 | P1 | P3 | P12, P13, and/or P14 | — | one or more of |
| | | | | | C1-C6, Q1, and/or |
| | | | | | S1 |
| 91 | P1 | P4 | P12, P13, and/or P14 | — | one or more of |
| | | | | | C1-C6, Q1, and/or |
| | | | | | S1 |
| 92 | P1 | P5 | P12, P13, and/or P14 | — | one or more of |
| | | | | | C1-C6, Q1, and/or |
| | | | | | S1 |
| 93 | P1 | P6 | P12, P13, and/or P14 | — | one or more of |
| | | | | | C1-C6, Q1, and/or |
| | | | | | S1 |
| 94 | P1 | P7 | P12, P13, and/or P14 | — | one or more of |
| | | | | | C1-C6, Q1, and/or |
| | | | | | S1 |
| 95 | P1 | P8 | P12, P13, and/or P14 | — | one or more of |
| | | | | | C1-C6, Q1, and/or |
| | | | | | S1 |
| 96 | P1 | P9 | P12, P13, and/or P14 | — | one or more of |
| | | | | | C1-C6, Q1, and/or |
| | | | | | S1 |
| 97 | P1 | P10 | P12, P13, and/or P14 | — | one or more of |
| | | | | | C1-C6, Q1, and/or |
| | | | | | S1 |
| 98 | P1 | one or more of P15-P28 | — | — | one or more of |
| | | | | | C1-C6, Q1, and/or |
| | | | | | S1 |
| 99 | P1 | one or more of P15-P28 | P11 | — | one or more of |
| | | | | | C1-C6, Q1, and/or |
| | | | | | S1 |
| 100 | P1 | one or more of P15-P28 | P12, P13, and/or P14 | — | one or more of |
| | | | | | C1-C6, Q1, and/or |
| | | | | | S1 |
| 101 | P1 | — | — | one or more | one or more of |
| | | | | of A1-A4 | C1-C6, Q1, and/or |
| | | | | | S1 |
| 102 | P1 | P2 | — | one or more | one or more of |
| | | | | of A1-A4 | C1-C6, Q1, and/or |
| | | | | | S1 |
| 103 | P1 | P3 | — | one or more | one or more of |
| | | | | of A1-A4 | C1-C6, Q1, and/or |
| | | | | | S1 |
| 104 | P1 | P4 | — | one or more | one or more of |
| | | | | of A1-A4 | C1-C6, Q1, and/or |
| | | | | | S1 |
| 105 | P1 | P5 | — | one or more | one or more of |
| | | | | of A1-A4 | C1-C6, Q1, and/or |
| | | | | | S1 |
| 106 | P1 | P6 | — | one or more | one or more of |
| | | | | of A1-A4 | C1-C6, Q1, and/or |
| | | | | | S1 |
| 107 | P1 | P7 | — | one or more | one or more of |
| | | | | of A1-A4 | C1-C6, Q1, and/or |
| | | | | | S1 |
| 108 | P1 | P8 | — | one or more | one or more of |
| | | | | of A1-A4 | C1-C6, Q1, and/or |
| | | | | | S1 |
| 109 | P1 | P9 | — | one or more | one or more of |
| | | | | of A1-A4 | C1-C6, Q1, and/or |
| | | | | | S1 |
| 110 | P1 | P10 | — | one or more | one or more of |
| | | | | of A1-A4 | C1-C6, Q1, and/or |
| | | | | | S1 |
| 111 | P1 | P11 | — | one or more | one or more of |
| | | | | of A1-A4 | C1-C6, Q1, and/or |
| | | | | | S1 |
| 112 | P1 | P2 | P11 | one or more | one or more of |
| | | | | of A1-A4 | C1-C6, Q1, and/or |
| | | | | | S1 |
| 113 | P1 | P3 | P11 | one or more | one or more of |
| | | | | of A1-A4 | C1-C6, Q1, and/or |
| | | | | | S1 |
| 114 | P1 | P4 | P11 | one or more | one or more of |
| | | | | of A1-A4 | C1-C6, Q1, and/or |
| | | | | | S1 |
| 115 | P1 | P5 | P11 | one or more | one or more of |
| | | | | of A1-A4 | C1-C6, Q1, and/or |
| | | | | | S1 |
| 116 | P1 | P6 | P11 | one or more | one or more of |
| | | | | of A1-A4 | C1-C6, Q1, and/or |
| | | | | | S1 |
| 117 | P1 | P7 | P11 | one or more | one or more of |
| | | | | of A1-A4 | C1-C6, Q1, and/or |
| | | | | | S1 |
| 118 | P1 | P8 | P11 | one or more | one or more of |
| | | | | of A1-A4 | C1-C6, Q1, and/or |
| | | | | | S1 |
| 119 | P1 | P9 | P11 | one or more | one or more of |
| | | | | of A1-A4 | C1-C6, Q1, and/or |
| | | | | | S1 |
| 120 | P1 | P10 | P11 | one or more | one or more of |
| | | | | of A1-A4 | C1-C6, Q1, and/or |
| | | | | | S1 |
| 121 | P1 | P12, P13, and/or P14 | — | one or more | one or more of |
| | | | | of A1-A4 | C1-C6, Q1, and/or |
| | | | | | S1 |
| 122 | P1 | P2 | P12, P13, and/or P14 | one or more | one or more of |
| | | | | of A1-A4 | C1-C6, Q1, and/or |
| | | | | | S1 |
| 123 | P1 | P3 | P12, P13, and/or P14 | one or more | one or more of |
| | | | | of A1-A4 | C1-C6, Q1, and/or |
| | | | | | S1 |
| 124 | P1 | P4 | P12, P13, and/or P14 | one or more | one or more of |
| | | | | of A1-A4 | C1-C6, Q1, and/or |
| | | | | | S1 |
| 125 | P1 | P5 | P12, P13, and/or P14 | one or more | one or more of |
| | | | | of A1-A4 | C1-C6, Q1, and/or |
| | | | | | S1 |
| 126 | P1 | P6 | P12, P13, and/or P14 | one or more | one or more of |
| | | | | of A1-A4 | C1-C6, Q1, and/or |
| | | | | | S1 |
| 127 | P1 | P7 | P12, P13, and/or P14 | one or more | one or more of |
| | | | | of A1-A4 | C1-C6, Q1, and/or |
| | | | | | S1 |
| 128 | P1 | P8 | P12, P13, and/or P14 | one or more | one or more of |
| | | | | of A1-A4 | C1-C6, Q1, and/or |
| | | | | | S1 |
| 129 | P1 | P9 | P12, P13, and/or P14 | one or more | one or more of |
| | | | | of A1-A4 | C1-C6, Q1, and/or |
| | | | | | S1 |
| 130 | P1 | P10 | P12, P13, and/or P14 | one or more | one or more of |
| | | | | of A1-A4 | C1-C6, Q1, and/or |
| | | | | | S1 |
| 131 | P1 | one or more of P15-P28 | — | one or more | one or more of |
| | | | | of A1-A4 | C1-C6, Q1, and/or |
| | | | | | S1 |
| 132 | P1 | one or more of P15-P28 | P11 | one or more | one or more of |
| | | | | of A1-A4 | C1-C6, Q1, and/or |
| | | | | | S1 |
| 133 | P1 | one or more of P15-P28 | P12, P13, and/or P14 | one or more | one or more of |
| | | | | of A1-A4 | C1-C6, Q1, and/or |
| | | | | | S1 |

### Methods of Use

The present invention also includes various methods of using 3-(heptyloxy)propane-1,2-diol to enhance one or more formulation characteristics. In some embodiments, the method is for preserving a composition, especially a cosmetic or pharmaceutical composition. In further embodiments, the method is for enhancing the rheology of (e.g., thickening) a composition, especially a cosmetic or pharmaceutical composition, thereby eliminating or minimizing usage levels of additional ingredients and lowering potential skin sensitizing effects. In general, these methods comprise mixing 3-(heptyloxy)propane-1,2-diol with at least one additional ingredient of the composition (e.g., an additional ingredient as described herein). In various embodiments, the invention is directed methods of enhancing the rheology of (thickening) a low-salt cosmetic composition, the method comprising mixing 3-(heptyloxy)propane-1,2-diol and 3-[(n-octyl)oxy]-1,2-propanediol with the composition, wherein the composition comprises 1 wt. % or less of inorganic salt. In various embodiments, the invention is directed to methods of producing a low-salt cosmetic composition that causes reduced irritation, the method comprising mixing 3-(heptyloxy)propane-1,2-diol and 3-[(n-octyl)oxy]-1,2-propanediol with the composition; wherein the composition comprises a lower percentage of inorganic salt to achieve a given viscosity than an otherwise identical composition lacking 3-(heptyloxy)propane-1,2-diol and/or 3-[(n-octyl)oxy]-1,2-propanediol.

In these methods, the composition can comprise any of the compositions described herein. In certain embodiments, the composition is selected from the group consisting of shampoo, conditioner, body wash, facial cleanser, and bubble bath.

The present invention is also directed to various methods of cleansing, treating, or caring for skin, hair, or teeth in a subject in need thereof using a composition as described herein. These methods comprise applying a composition as described in this application to the skin, hair, or teeth of the subject. In some embodiments, the composition is applied to an area of the subject selected from the group consisting of the face, neck, body, mouth, hand, foot, arm pit, around the eye, and combinations thereof. In certain embodiments, the method further comprises rinsing or removing the composition from the area.

The present invention is also directed to various methods of producing a preserved cosmetic composition, the method comprising: mixing 3-(heptyloxy)propane-1,2-diol and 3-[(n-octyl)oxy]-1,2-propanediol to form a preservative premix; and combining the preservative premix with the other components of the composition to form the preserved composition. Premixing the 3-(heptyloxy)propane-1,2-diol and 3-[(n-octyl)oxy]-1,2-propanediol reduces the amount of precipitate that forms in the final composition. Any of the other methods of producing compositions can mix the 3-(heptyloxy)propane-1,2-diol and 3-[(n-octyl)oxy]-1,2-propanediol together into a combination before the combination is mixed with the composition.

Having described the invention in detail, it will be apparent that modifications and variations are possible without departing from the scope of the invention defined in the appended claims.

### EXAMPLES

The following non-limiting examples are provided to further illustrate the present invention.

### Example 1: Formulations

The natural based compositions presented in Table 3 below were prepared and tested for stability. Given concentrations are percentages by weight, with water making up the remainder of the composition (indicated by Qs). Each type of composition had a low concentration formulation (with 0.5% 3-(heptyloxy)propane-1,2-diol) and a high concentration formulation (with 1% 3-(heptyloxy)propane-1,2-diol). The formulations were all found to exhibit suitable stability.

TABLE 3
Formulations
Natural cleansing wipe

| | | |
|---|---|---|
| Water | Aqua | Qs |
| Aloe Vera 200 | Aloe barbadensis Leaf Powder | 0.002 |
| Glycerine | Glycerin | 5 |
| APG 2000 | Decyl Glucoside | 4 |
| Sepiclear G7 | Heptyl Glucoside | 2.5 |
| 3-(heptyloxy)propane- | | 0.5 (low)- |
| 1,2-diol | | 1.0 (high) |
| Citric Acid | Citric Acid | pH 5.5 |

Emulsion wipe (Natural baby wipe)

| | | |
|---|---|---|
| Demineralized water | Aqua | Qs |
| Avicel PC 611 | Microcrystalline Cellulose(and) | 1.5 |
| | Cellulose Gum | |
| Glycerine | Glycerin | 5 |
| DUB 15P | Sucrose Palmitate | 1 |
| DUB SE 5S | Sucrose Distearate | 1 |
| Sesame Oil | Sesamum Indicum (Sesame) Seed Oil | 4.5 |
| Dermosoft Toco 70 | Helianthuus Annuus Seed Oil/Tocopherol | 0.5 |
| 3-(heptyloxy)propane- | | 0.5 (low)- |
| 1,2-diol | | 1.0 (high) |
| Citric Acid | Citric Acid | pH 5.5 |

Natural shampoo

| | | |
|---|---|---|
| Demineralized water | Aqua | Qs |
| DEHYQUART GUAR HP | Guar Hydroxypropyltrimonium Chloride | 0.25 |
| TEXAPON K30 | Sodium Coco Sulfate | 26.7 |
| PLANTACARE 2000 | Decyl Glucoside | 10 |
| LAMESOFT PO 65 | Coco-Glucoside/Glyceryl Oleate | 3 |
| NUTRILAN KERATIN LM | Hydrolyzed Keratine | 1 |
| CITRIC ACID | Citric Acid | pH 5.5 |
| 3-(heptyloxy)propane- | | 0.5 (low)- |
| 1,2-diol | | 1.0 (high) |
| Na Cl | Sodium Chloride | 1 |

Natural cream

| | | |
|---|---|---|
| Demineralized water | Aqua | Qs |
| Glycerine | Glycerin | 3 |
| KELTROL LAX-T | Xanthan Gum | 0.5 |
| Shea Butter | Butyrospermum Parkii (Shea) Butter | 2 |
| DUB MCT | Caprylic/Capric Triglycerides | 6 |
| DUB 810 C | Coco-Caprylate Caprate | 8 |
| Jojoba Oil | Simmondsia Chinensis Seed Oil | 2 |
| Plantasens Squalane | Squalane | 2 |
| Tocopherol CLR | Glycine Soja Oil/Tocopherol | 0.5 |
| Plantasens Natural | Sucrose Polystearate/Cetearyl Alcohol/ | 4 |
| Emulsifier HP 10 | Olea Europaea(Olive) Oil Unsaponifiables | |
| Ecorol 68/50 | Cetearyl Alcohol | 1.5 |
| Cotton extract | Glycerine/Aqua/Gossypium Herbaceum | 1 |
| | Seed Extract | |
| 3-(heptyloxy)propane- | | 0.5 (low)- |
| 1,2-diol | | 1.0 (high) |
| Citric Acid | Citric Acid | pH 5.5 |

Natural roll-on deodorant

| | | |
|---|---|---|
| AQUA | Aqua | Qs |
| Glycerine | Glycerine | 2 |
| Solagum AX | Acacia Senegal Gum/xanthan gum | 1 |
| ZEMEA | Propanediol | 5 |
| Montanov L | C₁₄₋₂₂ Alcohols (and) C₁₂₋₂₀ Alkyl | 2 |
| | Glucoside | |
| Ecorol 68/50 | Cetearyl Alcohol | 0.5 |
| EUTANOL G | Octyldodecanol | 2 |
| CETIOL OE | Dicaprylyl Ether | 3 |
| Cotton extract | Glycerine/Aqua/Gossypium Herbaceum | 1 |
| | Seed Extract | |
| 3-(heptyloxy)propane- | | 0.5 (low)- |
| 1,2-diol | | 1.0 (high) |
| Citric Acid | Citric Acid | pH 5.5 |

The procedure for making the natural cream formulation is as follows. Phase A is prepared by dispersing xanthan gum into water while stirring. Then, the glycerine is added. The mixture is continuously stirred until a homogeneous mixture is obtained without lumps. Phase A is then heated to 80-85° C. All of the remaining ingredients except for the 3-(heptyloxy)propane-1,2-diol and citric acid are added to the melter to form Phase B and heated to 80° C. Phase A and Phase B are emulsified by adding Phase B heated to 85° C. to Phase A for 5 minutes. The emulsion is then cooled with moderate stirring. 3-(heptyloxy)propane-1,2-diol and any other preservatives are then stirred in. Finally, the pH is adjusted to 5.5 if necessary with citric acid.

### Example 2: BIOLUMIX Assay to Screen for Preventing Microbial Growth

Efficacy of the compositions in preventing microbial growth were screened with a BIOLUMIX assay. The BIOLUMIX assay produces a color change when carbon dioxide, indicative of microbial contamination, is detected in a sample. Non-preserved formulations without 3-(heptyloxy)propane-1,2-diol were used as negative controls, and formulations were used at particular percentages of 3-(heptyloxy)propane-1,2-diol by weight. Passing criteria is meeting criteria A in Table 5. The results are shown in Table 4 below.

TABLE 4
BIOLUMIX Assay Results

| | CONCEN- | BIOLUMIX |
|---|---|---|
| DESCRIPTION | TRATION | RESULT |
| NATURAL CLEANSING WIPE-NON | | Failed |
| PRESERVED | | |
| NATURAL CLEANSING WIPE-3- | 0.50% | N/A |
| (heptyloxy)propane-1,2-diol-LOW | | |
| CONCENTRATION | | |
| NATURAL CLEANSING WIPE-3- | 1.00% | PASS |
| (heptyloxy)propane-1,2-diol-HIGH | | |
| CONCENTRATION | | |
| EMULSION WIPE-NON PRESERVED | | N/A |
| EMULSION WIPE-3- | 0.50% | N/A |
| (heptyloxy)propane-1,2-diol-LOW | | |
| CONCENTRATION | | |
| EMULSION WIPE-3- | 1.00% | PASS |
| (heptyloxy)propane-1,2-diol-HIGH | | |
| CONCENTRATION | | |
| NATURAL CREAM-NON | | N/A |
| PRESERVED | | |
| NATURAL CREAM-3- | 0.5% | N/A |
| (heptyloxy)propane-1,2-diol - LOW | | |
| CONCENTRATION | | |
| NATURAL CREAM-3- | 1% | PASS |
| (heptyloxy)propane-1,2-diol - HIGH | | |
| CONCENTRATION | | |
| NATURAL SHAMPOO-NON | | Failed |
| PRESERVED | | |
| NATURAL SHAMPOO-3- | 0.5% | N/A |
| (heptyloxy)propane-1,2-diol - LOW | | |
| CONCENTRATION | | |
| NATURAL SHAMPOO-3- | 1% | PASS |
| (heptyloxy)propane-1,2-diol - HIGH | | |
| CONCENTRATION | | |
| NATURAL ROLL-ON DEODORANT - | | N/A |
| NON PRESERVED | | |
| NATURAL ROLL-ON DEODORANT- | 1.00% | PASS |
| HIGH CONCENTRATION | | |

As expected, the non-preserved cleansing wipe and natural shampoo samples failed the BIOLUMIX assay, which is indicative of microbial growth within these samples without the presence of a preservative. However, the cleansing wipe, emulsion wipe, natural cream, natural shampoo, and natural roll-on deodorant formulations used at a 1% by weight dosage passed the BIOLUMIX assay, indicating 3-(heptyloxy)propane-1,2-diol successfully blocks microbial growth in these products that would normally get contaminated.

### Example 3. Challenge Tests

Select formulations prepared in accordance with Example 1 (i.e., natural cleansing wipe, emulsion wipe, and natural roll-on deodorant) were subjected to preservative challenge testing for antimicrobial effectiveness (CTNF). The testing was performed in accordance with ISO 11930. The ISO 11930 methodology is similar to USP-NF<51> and evaluates the activity of preservatives or other intrinsic characteristics of a product that help maintain the safety of a product by inhibiting the growth and reducing the amount of microbial contaminants. The CTNF testing requirements used as standards in the challenge tests are shown in Table 5 below. The formulations used in the challenge tests are described in Tables 3 and 4 above.

TABLE 5
CTNF Testing Requirements
Rate of logarithmic reduction required ^{a}

| | Microorganisms | | | | | | | |
|---|---|---|---|---|---|---|---|---|
| | BACTERIA | | | YEASTS | | | MOLDS | |
| Time | D 7 | D 14 | D 28 | D 7 | D 14 | D 28 | D 14 | D 28 |
| Criteria A | ≥3 | ≥3 | ≥3 | ≥1 | ≥1 | >1 | ≥0 ^{c} | ≥1 |
| | | and | and | | and | and | | |
| | | NI ^{b} | NI | | NI | NI | | |
| Criteria B | Not | ≥3 | ≥3 | Not | ≥1 | ≥1 | ≥0 | >0 |
| | realized | | and | realized | | and | | and |
| | | | NI | | | NI | | NI |

^{a} In this study, an incertitude of 0.5 log is acceptable.
^{b} NI: no increase of microorganisms than the previously time of contact.
^{c} No increase than the initial number.

Non-preserved formulations without 3-(heptyloxy)propane-1,2-diol were used as negative controls, and formulations were used at particular percentages of 3-(heptyloxy)propane-1,2-diol by weight. Formulations were tested against Staphylococcus aureus, Pseudomonas aeruginosa, Escherichia coli, Candida albicans and Aspergillus brasiliensis. The results for the logarithmic reduction in contamination by particular species at days 7, 14, and 28 are shown in the below tables.

TABLE 6
Natural Cleansing Wipe Challenge Tests
Obtained reductions (logarithmic)

| Cleansing wipe type | Strain/type of challenge | | Day 7 | Day 14 | Day 28 |
|---|---|---|---|---|---|
| Non-preserved | Staphylococcus | Bacteria | >3.30 | >3.30 | >3.30 |
| | aureus | | | | |
| Non-preserved | Pseudomonas | Bacteria | 0.00 | 0.00 | 0.00 |
| | aeruginosa | | | | |
| Non-preserved | Escherichia coli | Bacteria | >3.41 | >3.41 | >3.41 |
| Non-preserved | Candida albicans | Fungi (yeast) | 1.72 | 0.34 | 0.96 |
| Non-preserved | Aspergillus | Fungi (mold) | 1.70 | 1.22 | 0.22 |
| | brasiliensis | | | | |
| Low concentration | Staphylococcus | Bacteria | >3.30 | >3.30 | >3.30 |
| (0.5%) | aureus | | | | |
| Low concentration | Pseudomonas | Bacteria | >3.34 | >3.34 | >3.34 |
| (0.5%) | aeruginosa | | | | |
| Low concentration | Escherichia coli | Bacteria | >3.41 | >3.41 | >3.41 |
| (0.5%) | | | | | |
| Low concentration | Candida albicans | Fungi (yeast) | >2.32 | >2.32 | >2.32 |
| (0.5%) | | | | | |
| Low concentration | Aspergillus | Fungi (mold) | 1.44 | 2.40 | >2.40 |
| (0.5%) | brasiliensis | | | | |
| High concentration (1%) | Staphylococcus | Bacteria | >3.30 | >3.30 | >3.30 |
| | aureus | | | | |
| High concentration (1%) | Pseudomonas | Bacteria | >3.34 | >3.34 | >3.34 |
| | aeruginosa | | | | |
| High concentration (1%) | Escherichia coli | Bacteria | >3.41 | >3.41 | >3.41 |
| High concentration (1%) | Candida albicans | Fungi (yeast) | >2.32 | >2.32 | >2.32 |
| High concentration (1%) | Aspergillus | Fungi (mold) | 0.75 | 2.40 | >2.40 |
| | brasiliensis | | | | |

TABLE 7
Emulsion Wipe Challenge Tests
Obtained reductions (logarithmic)

| Emulsion wipe type | Strain/type of challenge | | Day 7 | Day 14 | Day 28 |
|---|---|---|---|---|---|
| Low concentration | Staphylococcus | Bacteria | >3.20 | >3.20 | >3.20 |
| (0.5%) | aureus | | | | |
| Low concentration | Pseudomonas | Bacteria | >3.34 | >3.34 | >3.34 |
| (0.5%) | aeruginosa | | | | |
| Low concentration | Escherichia coli | Bacteria | >3.32 | >3.32 | >3.32 |
| (0.5%) | | | | | |
| Low concentration | Candida albicans | Fungi (yeast) | >2.40 | >2.40 | >2.40 |
| (0.5%) | | | | | |
| Low concentration | Aspergillus | Fungi (mold) | 0.00 | 0.00 | 1.37 |
| (0.5%) | brasiliensis | | | | |
| High concentration (1%) | Staphylococcus | Bacteria | >3.20 | >3.20 | >3.20 |
| | aureus | | | | |
| High concentration (1%) | Pseudomonas | Bacteria | >3.34 | >3.34 | >3.34 |
| | aeruginosa | | | | |
| High concentration (1%) | Escherichia coli | Bacteria | >3.32 | >3.32 | >3.32 |
| High concentration (1%) | Candida albicans | Fungi (yeast) | >2.40 | >2.40 | >2.40 |
| High concentration (1%) | Aspergillus | Fungi (mold) | 0.10 | 0.00 | 1.07 |
| | brasiliensis | | | | |

The results indicate that formulations containing 3-(heptyloxy)propane-1,2-diol are all approved via CTNF testing, meeting the Criteria A requirements of antimicrobial protection performance. As expected, the negative control does not meet the Criteria A requirements. Thus, the microbiological risk of these 3-(heptyloxy)propane-1,2-diol-containing formulations is considered acceptable and the cosmetic product meets the requirements of ISO 11930:2019.

The results of cell count for the contamination by particular species at days 0, 7, 14, and 28 are shown in the below tables.

TABLE 8
Natural Cream Challenge Tests

| Natural | | Type of | Cell Count | Cell Count | Cell Count | Cell Count |
|---|---|---|---|---|---|---|
| Cream Type | Strain | Challenge | Day 0 | Day 7 | Day 14 | Day 28 |
| Low | Staphylococcus | Bacteria | 1.30 × 10⁵ | <1.0 × 10² | <1.0 × 10² | <1.0 × 10² |
| concentration | aureus | | | | | |
| (0.5%) | | | | | | |
| Low | Pseudomonas | Bacteria | 2.80 × 10⁵ | <1.0 × 10² | <1.0 × 10² | <1.0 × 10² |
| concentration | aeruginosa | | | | | |
| (0.5%) | | | | | | |
| Low | Escherichia | Bacteria | 1.50 × 10⁵ | <1.0 × 10² | <1.0 × 10² | <1.0 × 10² |
| concentration | coli | | | | | |
| (0.5%) | | | | | | |
| Low | Candida | Fungi | 1.50 × 10⁴ | <1.0 × 10² | <1.0 × 10² | <1.0 × 10² |
| concentration | albicans | (yeast) | | | | |
| (0.5%) | | | | | | |
| Low | Aspergillus | Fungi | 4.40 × 10⁴ | 1.90 × 10⁴ | 4.40 × 10⁴ | 1.40 × 10⁴ |
| concentration | brasiliensis | (mold) | | | | |
| (0.5%) | | | | | | |
| High | Staphylococcus | Bacteria | 1.30 × 10⁵ | <1.0 × 10² | <1.0 × 10² | <1.0 × 10² |
| concentration | aureus | | | | | |
| (1%) | | | | | | |
| High | Pseudomonas | Bacteria | 2.80 × 10⁵ | <1.0 × 10² | <1.0 × 10² | <1.0 × 10² |
| concentration | aeruginosa | | | | | |
| (1%) | | | | | | |
| High | Escherichia | Bacteria | 1.50 × 10⁵ | <1.0 × 10² | <1.0 × 10² | <1.0 × 10² |
| concentration | coli | | | | | |
| (1%) | | | | | | |
| High | Candida | Fungi | 1.50 × 10⁴ | <1.0 × 10² | <1.0 × 10² | <1.0 × 10² |
| concentration | albicans | (yeast) | | | | |
| (1%) | | | | | | |
| High | Aspergillus | Fungi | 4.40 × 10⁴ | 4.30 × 10³ | 2.10 × 10³ | 1.00 × 10² |
| concentration | brasiliensis | (mold) | | | | |
| (1%) | | | | | | |

TABLE 9
Natural Shampoo Challenge Tests

| Natural | | | | | | |
|---|---|---|---|---|---|---|
| Shampoo | | Type of | Cell Count | Cell Count | Cell Count | Cell Count |
| Type | Strain | Challenge | Day 0 | Day 7 | Day 14 | Day 28 |
| Non- | Staphylococcus | Bacteria | 1.30 × 10⁵ | <1.0 × 10² | <1.0 × 10² | <1.0 × 10² |
| preserved | aureus | | | | | |
| Non- | Pseudomonas | Bacteria | 2.80 × 10⁵ | 1.40 × 10³ | <1.0 × 10² | <1.0 × 10² |
| preserved | aeruginosa | | | | | |
| Non- | Escherichia | Bacteria | 1.50 × 10⁵ | 1.50 × 10⁵ | 1.50 × 10⁵ | 1.50 × 10⁵ |
| preserved | coli | | | | | |
| Non- | Candida | Fungi | 1.50 × 10⁴ | <1.0 × 10² | <1.0 × 10² | <1.0 × 10² |
| preserved | albicans | (yeast) | | | | |
| Non- | Aspergillus | Fungi | 4.40 × 10⁴ | 1.70 × 10⁴ | 1.50 × 10⁴ | 5.70 × 10³ |
| preserved | brasiliensis | (mold) | | | | |
| Low | Staphylococcus | Bacteria | 1.30 × 10⁵ | <1.0 × 10² | <1.0 × 10² | <1.0 × 10² |
| concentration | aureus | | | | | |
| (0.5%) | | | | | | |
| Low | Pseudomonas | Bacteria | 2.80 × 10⁵ | 3.80 × 10⁴ | 2.00 × 10² | 2.00 × 10² |
| concentration | aeruginosa | | | | | |
| (0.5%) | | | | | | |
| Low | Escherichia | Bacteria | 1.50 × 10⁵ | 1.50 × 10⁵ | 1.50 × 10⁵ | 1.50 × 10⁵ |
| concentration | coli | | | | | |
| (0.5%) | | | | | | |
| Low | Candida | Fungi | 1.50 × 10⁴ | <1.0 × 10² | <1.0 × 10² | <1.0 × 10² |
| concentration | albicans | (yeast) | | | | |
| (0.5%) | | | | | | |
| Low | Aspergillus | Fungi | 4.40 × 10⁴ | 1.80 × 10⁴ | 2.10 × 10⁴ | 1.50 × 10⁴ |
| concentration | brasiliensis | (mold) | | | | |
| (0.5%) | | | | | | |
| High | Staphylococcus | Bacteria | 1.30 × 10⁵ | <1.0 × 10² | <1.0 × 10² | <1.0 × 10² |
| concentration | aureus | | | | | |
| (1%) | | | | | | |
| High | Pseudomonas | Bacteria | 2.80 × 10⁵ | <1.0 × 10² | <1.0 × 10² | <1.0 × 10² |
| concentration | aeruginosa | | | | | |
| (1%) | | | | | | |
| High | Escherichia | Bacteria | 1.50 × 10⁵ | 1.50 × 10⁵ | 1.50 × 10⁵ | 1.50 × 10⁵ |
| concentration | coli | | | | | |
| (1%) | | | | | | |
| High | Candida | Fungi | 1.50 × 10⁴ | <1.0 × 10² | <1.0 × 10² | <1.0 × 10² |
| concentration | albicans | (yeast) | | | | |
| (1%) | | | | | | |
| High | Aspergillus | Fungi | 4.40 × 10⁴ | 1.90 × 10⁴ | 4.10 × 10⁴ | 2.00 × 10⁴ |
| concentration | brasiliensis | (mold) | | | | |
| (1%) | | | | | | |

The roll-on deodorant challenge tests shown below were conducted in accordance with ISO 11930 except that formulations were tested with bacteria Corynebacterium xerosis—code souchier M′ and Staphylococcus epidermidis—code souchier C′, which are two of the major odor-causing bacteria found in skin flora. The roll-on deodorant formulations used in the challenge tests are described in Tables 3 and 4 above. A non-preserved formulation without 3-(heptyloxy)propane-1,2-diol was used as a negative control, and the high concentration formulation with 1% 3-(heptyloxy)propane-1,2-diol by weight was tested. The results for the logarithmic reduction in contamination by particular species at days 7, 14, and 28 are shown in Table 10.

TABLE 10
Roll-On Deodorant Challenge Tests
Obtained reductions (logarithmic)

| Roll-on | | | | | |
|---|---|---|---|---|---|
| deodorant type | Strain/type of challenge | | Day 7 | Day 14 | Day 28 |
| Non-preserved | Corynebacterium | Bacteria | >3.11 | >3.11 | >3.11 |
| | xerosis - code | | | | |
| | souchier M′ | | | | |
| Non-preserved | Staphylococcus | Bacteria | 1.33 | >3.56 | >3.56 |
| | epidermidis - | | | | |
| | code souchier C′ | | | | |
| High | Corynebacterium | Bacteria | >3.11 | >3.11 | >3.11 |
| concentration | xerosis - code | | | | |
| (1%) | souchier M′ | | | | |
| High | Staphylococcus | Bacteria | >3.56 | >3.56 | >3.56 |
| concentration | epidermidis - | | | | |
| (1%) | code souchier C′ | | | | |

This demonstrates that addition of 1% 3-(heptyloxy)propane-1,2-diol increases logarithmic reduction of bacteria such as Staphylococcus faster in natural roll-on deodorant.

### Example 4. BIOLUMIX Tests with Multiple Preservatives

The following example demonstrates the effect of 3-(heptyloxy)propane-1,2-diol and 3-[(n-octyl)oxy]-1,2-propanediol in combination as a preservative. The formulations are the same as in the previous Examples, except 3-(heptyloxy)propane-1,2-diol and 3-[(n-octyl)oxy]-1,2-propanediol are both added as preservatives. The BIOLUMIX tests were conducted in the same way, and the percentages reported in the tables below are the percentages of each preservative added to that particular formulation.

TABLE 11
Preservation of Natural Cream with 3-(heptyloxy)propane-
1,2-diol and 3-[(n-octyl)oxy]-1,2-propanediol

| Natural | | Type of | Cell Count | Cell Count | Cell Count |
|---|---|---|---|---|---|
| Cream Type | Strain | Challenge | Day 0 | Day 7 | Day 14 |
| Low | Staphylococcus | Bacteria | 1.60 × 10⁵ | <1.0 × 10² | <1.0 × 10² |
| concentration | aureus | | | | |
| (0.25%) | | | | | |
| Low | Pseudomonas | Bacteria | 3.10 × 10⁵ | <1.0 × 10² | <1.0 × 10² |
| concentration | aeruginosa | | | | |
| (0.25%) | | | | | |
| Low | Escherichia | Bacteria | 2.30 × 10⁵ | <1.0 × 10² | <1.0 × 10² |
| concentration | coli | | | | |
| (0.25%) | | | | | |
| Low | Candida | Fungi | 4.00 × 10⁴ | <1.0 × 10² | <1.0 × 10² |
| concentration | albicans | (yeast) | | | |
| (0.25%) | | | | | |
| Low | Aspergillus | Fungi | 5.10 × 10⁴ | 3.00 × 10⁴ | 2.80 × 10⁴ |
| concentration | brasiliensis | (mold) | | | |
| (0.25%) | | | | | |
| High | Staphylococcus | Bacteria | 1.60 × 10⁵ | <1.0 × 10² | <1.0 × 10² |
| concentration | aureus | | | | |
| (0.5%) | | | | | |
| High | Pseudomonas | Bacteria | 3.10 × 10⁵ | <1.0 × 10² | <1.0 × 10² |
| concentration | aeruginosa | | | | |
| (0.5%) | | | | | |
| High | Escherichia | Bacteria | 2.30 × 10⁵ | <1.0 × 10² | <1.0 × 10² |
| concentration | coli | | | | |
| (0.5%) | | | | | |
| High | Candida | Fungi | 4.00 × 10⁴ | <1.0 × 10² | <1.0 × 10² |
| concentration | albicans | (yeast) | | | |
| (0.5%) | | | | | |
| High | Aspergillus | Fungi | 5.10 × 10⁴ | 3.60 × 10³ | 1.40 × 10³ |
| concentration | brasiliensis | (mold) | | | |
| (0.5%) | | | | | |

TABLE 12
Preservation of Emulsion Wipe with 3-(heptyloxy)propane-
1,2-diol and 3-[(n-octyl)oxy]-1,2-propanediol

| Emulsion | | Type of | Cell Count | Cell Count | Cell Count |
|---|---|---|---|---|---|
| Wipe Type | Strain | Challenge | Day 0 | Day 7 | Day 14 |
| Low | Staphylococcus | Bacteria | 1.60 × 10⁵ | <1.0 × 10² | <1.0 × 10² |
| concentration | aureus | | | | |
| (0.25%) | | | | | |
| Low | Pseudomonas | Bacteria | 3.10 × 10⁵ | <1.0 × 10² | <1.0 × 10² |
| concentration | aeruginosa | | | | |
| (0.25%) | | | | | |
| Low | Escherichia | Bacteria | 2.30 × 10⁵ | <1.0 × 10² | <1.0 × 10² |
| concentration | coli | | | | |
| (0.25%) | | | | | |
| Low | Candida | Fungi | 4.00 × 10⁴ | <1.0 × 10² | <1.0 × 10² |
| concentration | albicans | (yeast) | | | |
| (0.25%) | | | | | |
| Low | Aspergillus | Fungi | 5.10 × 10⁴ | 3.00 × 10⁴ | 2.80 × 10⁴ |
| concentration | brasiliensis | (mold) | | | |
| (0.25%) | | | | | |
| High | Staphylococcus | Bacteria | 1.60 × 10⁵ | <1.0 × 10² | <1.0 × 10² |
| concentration | aureus | | | | |
| (0.5%) | | | | | |
| High | Pseudomonas | Bacteria | 3.10 × 10⁵ | <1.0 × 10² | <1.0 × 10² |
| concentration | aeruginosa | | | | |
| (0.5%) | | | | | |
| High | Escherichia | Bacteria | 2.30 × 10⁵ | 8.6 × 10⁴ | <1.0 × 10² |
| concentration | coli | | | | |
| (0.5%) | | | | | |
| High | Candida | Fungi | 4.00 × 10⁴ | <1.0 × 10² | <1.0 × 10² |
| concentration | albicans | (yeast) | | | |
| (0.5%) | | | | | |
| High | Aspergillus | Fungi | 5.10 × 10⁴ | 2.40 × 10⁴ | 5.50 × 10³ |
| concentration | brasiliensis | (mold) | | | |
| (0.5%) | | | | | |

TABLE 13
Preservation of Natural Shapoo with 3-(heptyloxy)propane-
1,2-diol and 3-[(n-octyl)oxy]-1,2-propanediol

| Natural | | | | | |
|---|---|---|---|---|---|
| Shampoo | | Type of | Cell Count | Cell Count | Cell Count |
| Type | Strain | Challenge | Day 0 | Day 7 | Day 14 |
| Non- | Staphylococcus | Bacteria | 2.00 × 10⁵ | <1.0 × 10² | <1.0 × 10² |
| preserved | aureus | | | | |
| Non- | Pseudomonas | Bacteria | 1.40 × 10⁵ | <1.0 × 10² | <1.0 × 10² |
| preserved | aeruginosa | | | | |
| Non- | Escherichia | Bacteria | 1.30 × 10⁵ | 1.30 × 10⁵ | 1.30 × 10⁵ |
| preserved | coli | | | | |
| Non- | Candida | Fungi | 1.60 × 10⁴ | <1.0 × 10² | <1.0 × 10² |
| preserved | albicans | (yeast) | | | |
| Non- | Aspergillus | Fungi | 1.50 × 10⁴ | <1.0 × 10² | <1.0 × 10² |
| preserved | brasiliensis | (mold) | | | |
| Low | Staphylococcus | Bacteria | 1.60 × 10⁵ | <1.0 × 10² | <1.0 × 10² |
| concentration | aureus | | | | |
| (0.25%) | | | | | |
| Low | Pseudomonas | Bacteria | 3.10 × 10⁵ | <1.0 × 10² | <1.0 × 10² |
| concentration | aeruginosa | | | | |
| (0.25%) | | | | | |
| Low | Escherichia | Bacteria | 2.30 × 10⁵ | <1.0 × 10² | <1.0 × 10² |
| concentration | coli | | | | |
| (0.25%) | | | | | |
| Low | Candida | Fungi | 4.00 × 10⁴ | <1.0 × 10² | <1.0 × 10² |
| concentration | albicans | (yeast) | | | |
| (0.25%) | | | | | |
| Low | Aspergillus | Fungi | 5.10 × 10⁴ | 1.70 × 10⁴ | 8.40 × 10³ |
| concentration | brasiliensis | (mold) | | | |
| (0.25%) | | | | | |
| High | Staphylococcus | Bacteria | 1.60 × 10⁵ | <1.0 × 10² | <1.0 × 10² |
| concentration | aureus | | | | |
| (0.5%) | | | | | |
| High | Pseudomonas | Bacteria | 3.10 × 10⁵ | <1.0 × 10² | <1.0 × 10² |
| concentration | aeruginosa | | | | |
| (0.5%) | | | | | |
| High | Escherichia | Bacteria | 2.30 × 10⁵ | <1.0 × 10² | <1.0 × 10² |
| concentration | coli | | | | |
| (0.5%) | | | | | |
| High | Candida | Fungi | 4.00 × 10⁴ | <1.0 × 10² | <1.0 × 10² |
| concentration | albicans | (yeast) | | | |
| (0.5%) | | | | | |
| High | Aspergillus | Fungi | 5.10 × 10⁴ | 1.60 × 10⁴ | 6.80 × 10³ |
| concentration | brasiliensis | (mold) | | | |
| (0.5%) | | | | | |

The non-preserved formulation failed. The natural cream low and high concentrations, emulsion wipe low concentration, and natural shampoo low and high concentrations passed the A requirements. The emulsion wipe high concentration passed the B requirements.

### Example 5. Synergistic Thickening with 3-(heptyloxy)propane-1,2-diol and 3-[(n-octyl)oxy]-1,2-propanediol

The following example demonstrates the effect of 3-(heptyloxy)propane-1,2-diol, and 3-[(n-octyl)oxy]-1,2-propanediol on rheology/thickening in various surfactant systems. Four formulations are shown in the four rightmost columns of Table 14 with ingredients indicated as a percentage by weight. The formulations differ in percentages of sodium chloride, 3-(heptyloxy)propane-1,2-diol, and 3-[(n-octyl)oxy]-1,2-propanediol. The resulting viscosities and relative changes in viscosity are shown in the bottom two rows of the table.

TABLE 14
3-(heptyloxy)propane-1,2-diol/3-[(n-octyl)oxy]-1,2-propanediol/surfactant viscosity formulation

| | | Formulations | | | |
|---|---|---|---|---|---|
| | Ingredient Synonym | 1 | 2 | 3 | 4 |
| Ingredient | | | | | |
| Demin. Water | Aqua | 59.05 | 58.05 | 57.05 | 57.05 |
| Sulfopon 1216G | Sodium Coco Sulfate | 8.00 | 8.00 | 8.00 | 8.00 |
| Dehyquart Guar HP | Guar Hydroxypropyltrimonium | 0.25 | 0.25 | 0.25 | 0.25 |
| | Chloride | | | | |
| Plantacare 2000 | Decyl Glucoside (50%) | 10 | 10 | 10 | 10 |
| Lamesoft PO65 | Coco-Glucoside (and) Glyceryl | 3 | 3 | 3 | 3 |
| | Oleate | | | | |
| Nutrilan Keratin | Hydrolyzed Keratin | 1 | 1 | 1 | 1 |
| sodium chloride | | 0 | 1 | 1 | 1 |
| 3-(heptyloxy)propane-1,2-diol | | 0 | 0 | 1 | 1 |
| 3-[(n-octyl)oxy]-1,2- | Caprylyl Glyceryl Ether | 0 | 0 | 0 | 1 |
| propanediol | | | | | |
| citric acid | Citric Acid | pH 5.5 | pH 5.5 | pH 5.5 | pH 5.5 |
| Properties | | | | | |
| Change in viscosity | | | ↑ | ↑+ | ↑++ |
| Viscosity | LV3/12 rpm at room | 70 cps | 280 cps | 1600 cps | 4380 cps |
| | temperature (Brookfield | | | | |
| | Rheometer) | | | | |

This example demonstrates that the addition of 1% 3-(heptyloxy)propane-1,2-diol and 1% 3-[(n-octyl)oxy]-1,2-propanediol considerably increases the viscosity of the surfactant system.

A thickening behavior study was also performed in another basic surfactant system: Anionic/Non Ionic (APG—alkyl polyglucoside). Each column corresponds to a particular formulation with ingredients listed as a percentage by weight. The remainder was water, represented by Qs. % AM indicates % of active matter (e.g. % of dry surfactant). The viscosities for each formulation are bolded. All formulations had a clear appearance.

TABLE 15
Thickening behavior study in a basic surfactant system: Anionic*/Non Ionic (APG)

| | Na Cocoyl Sulfate | | | | Ammonium Lauryl Sulfate | | | | Na Lauryl Ether Sulfate | | | |
|---|---|---|---|---|---|---|---|---|---|---|---|---|
| 12% AM | (SCS)/APG System | | | | (ALS)/APG System | | | | (SLES)/APG System | | | |
| Aqua | Qs | Qs | Qs | Qs | Qs | Qs | Qs | Qs | Qs | Qs | Qs | Qs |
| *SCS | 8 | 8 | 8 | 8 | — | — | — | — | — | — | — | — |
| ALS | — | — | — | — | 8 | 8 | 8 | 8 | — | — | — | — |
| SLES | — | — | — | — | — | — | — | — | 8 | 8 | 8 | 8 |
| APG | 4 | 4 | 4 | 4 | 4 | 4 | 4 | 4 | 4 | 4 | 4 | 4 |
| Citric Acid | pH 5.5 | pH 5.5 | pH 5.5 | pH 5.5 | pH 5.5 | pH 5.5 | pH 5.5 | pH 5.5 | pH 5.5 | pH 5.5 | pH 5.5 | pH 5.5 |
| NaCl | 1 | 1 | 1 | 1 | 1 | 1 | 1 | 1 | 1 | 1 | 1 | 1 |
| Viscosity | 50 cps | | | | 50 cps | | | | 40 cps | | | |
| 3- | | 1 | | 1 | | 1 | | 1 | | 1 | | 1 |
| (heptyloxy) | | | | | | | | | | | | |
| propane- | | | | | | | | | | | | |
| 1,2-diol | | | | | | | | | | | | |
| Viscosity | | 130 cps | | | | 260 cps | | | | 50 cps | | |
| 3-[(n- | | | 1 | 1 | | | 1 | 1 | | | 1 | 1 |
| octyl)oxy]- | | | | | | | | | | | | |
| 1,2- | | | | | | | | | | | | |
| propanediol | | | | | | | | | | | | |
| Viscosity | | | 370 cps | 1440 cps | | | 550 cps | 1560 cps | | | 50 cps | 110 cps |

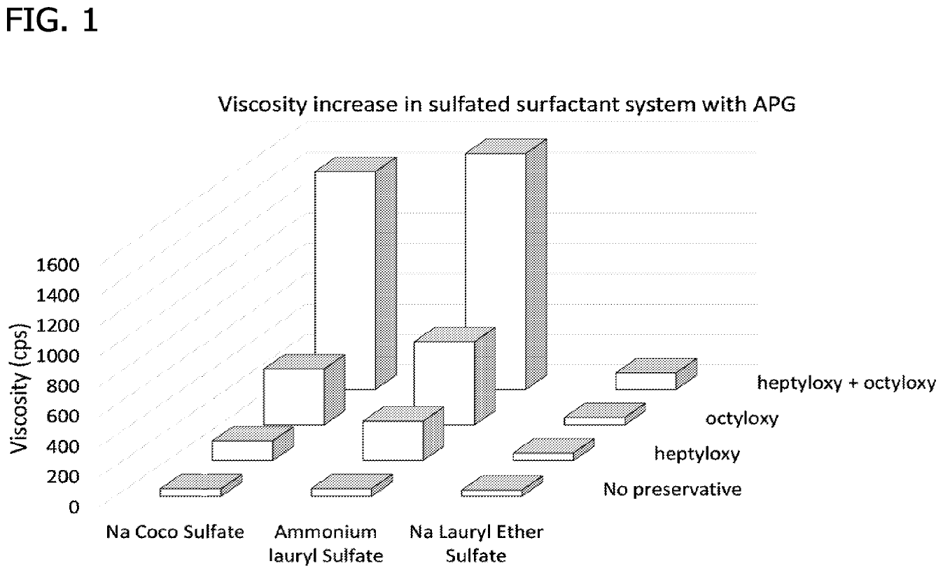

All formulations had a clear appearance. This testing demonstrates that 3-(heptyloxy)propane-1,2-diol and 3-[(n-octyl)oxy]-1,2-propanediol provide for an unexpected increase in viscosity (i.e., thickening) in a sulfated surfactant system with APG. The results of Table 15 are depicted in FIG. 1.

A further thickening behavior study was performed in a different basic surfactant system: anionic/amphoteric (CAPB—Cocamidopropyl betaine). The results are shown in Table 16 below. Each column corresponds to a particular formulation with ingredients listed as a percentage by weight. The remainder was water, represented by Qs. % AM indicates % of active matter (e.g. % of dry surfactant). The viscosities for each formulation are bolded.

TABLE 16
Thickening behavior study in a basic surfactant system: Anionic*/Amphoteric (CAPB)

| | Na Cocoyl | | | | | | | | |
|---|---|---|---|---|---|---|---|---|---|
| | Sulfate | | | | | | | | |
| | (SCS)/CAPB | Ammonium Lauryl Sulfate (ALS)/ | | | | Na Lauryl Ether Sulfate (SLES)/ | | | |
| 12% AM | System | CAPB System | | | | CAPB System | | | |
| Aqua | Qs | Qs | Qs | Qs | Qs | Qs | Qs | Qs | Qs |
| *SCS | 10 | — | — | — | — | — | — | — | — |
| ALS | — | 10 | 10 | 10 | 10 | — | — | — | — |
| SLES | — | — | — | — | — | 10 | 10 | 10 | 10 |
| CAPB | 2 | 2 | 2 | 2 | 2 | 2 | 2 | 2 | 2 |
| Citric Acid | pH 5.5 | pH 5.5 | pH 5.5 | pH 5.5 | pH 5.5 | pH 5.5 | pH 5.5 | pH 5.5 | pH 5.5 |
| NaCl | 1 | 1 | 1 | 1 | 1 | 1 | 1 | 1 | 1 |
| Viscosity | solid | 600 cps | | | | 50 cps | | | |
| 3- | | | 1 | | 1 | | 1 | | 1 |
| (heptyloxy)propane- | | | | | | | | | |
| 1,2-diol | | | | | | | | | |
| Viscosity | | | 2500 cps | | | | 200 cps | | |
| 3-[(n-octyl)oxy]- | | | | 1 | 1 | | | 1 | 1 |
| 1,2-propanediol | | | | | | | | | |
| Viscosity | | | | 3250 cps | 5780 cps | | | 700 cps | 4160 cps |

The Na cocoyl sulfate/CAPB formulation became solid at room temperature and thus was not usable.

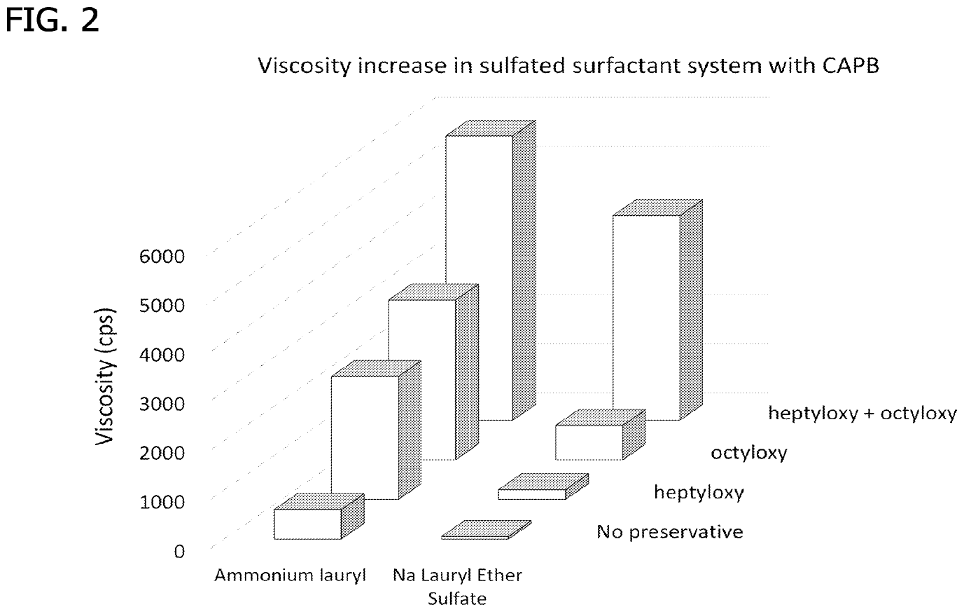

The ammonium lauryl sulfate/CAPB and Na lauryl ether sulfate/CAPB formulations all had a clear appearance. These results show that when used together 3-(heptyloxy)propane-1,2-diol and 3-[(n-octyl)oxy]-1,2-propanediol in a surfactant system with an amphoteric surfactant provide for an unexpected synergistic increase in viscosity (i.e., thickening). The results of Table 16 are depicted in FIG. 2.

A further thickening behavior study was performed to compare sulfated surfactants and sulfate free surfactants. The results are shown in Table 17 below. Each column corresponds to a particular formulation with ingredients listed as a percentage by weight. The remainder was water, represented by Qs. % AM indicates % of active matter (e.g. % of dry surfactant). Sodium chloride, 3-(heptyloxy)propane-1,2-diol, and 3-[(n-octyl)oxy]-1,2-propanediol were added sequentially with the change in viscosity measured after each addition and noted in Table 17 below.

TABLE 17

| | Sulfated Surfactants | | | | Sulfate Free Surfactants | | | | |
|---|---|---|---|---|---|---|---|---|---|
| | Na | Na | Ammonium | Na Lauryl | | | | | Na Lauryl |
| | Coco | Lauryl | Lauryl | Ether | Na Cocoyl | Na Cocoyl | Na Lauroyl | Na Lauryl | Glucose |
| | Sulfate | Sulfate | Sulfate | Sulfate | Glutamate | Glycinate | Sarcosinate | Sulfoacetate | Carboxylate |
| 12% AM | % w/w | % w/w | % w/w | % w/w | % w/w | % w/w | % w/w | % w/w | % w/w |
| Aqua | Qs | Qs | Qs | Qs | Qs | Qs | Qs | Qs | Qs |
| Anionic Surfactant | 8 | 8 | 8 | 8 | 8 | 8 | 8 | 8 | 8 |
| Decyl Glucoside | 4 | 4 | 4 | 4 | 4 | 4 | 4 | 4 | 4 |
| Citric Acid | pH 5.5 | pH 5.5 | pH 5.5 | pH 5.5 | pH 5.5 | pH 5.5 | pH 5.5 | pH 5.5 | pH 5.5 |
| NaCl | 1 | 1 | 1 | 1 | 2 | 2 | 2 | 2 | 2 |
| Viscosity | ↑ | ↑ | ↑ | ↑ | ≈ | ↑ | ≈ | ≈ | ≈ |
| 3-(heptyloxy)propane- | 1 | 1 | 1 | 1 | 1 | 1 | 1 | 1 | 1 |
| 1,2-diol | | | | | | | | | |
| Viscosity | ↑+ | ↑ | ↑+ | ↑+ | ≈ | ≈ | ≈ | ≈ | ≈ |
| 3-[(n-octyl)oxy]-1,2- | 1 | 1 | 1 | 1 | 1 | 1 | 1 | 1 | 1 |
| propanediol | | | | | | | | | |
| Viscosity | ↑++ | ↑++ | ↑++ | ↑++ | ≈ | ≈ | ≈ | ≈ | ≈ |
| Appearance | Clear | Clear | Clear | Clear | Clear | White | Clear | Hazy | Hazy |

Table 17 demonstrates that 1% 3-(heptyloxy)propane-1,2-diol and 1% 3-[(n-octyl)oxy]-1,2-propanediol can build up viscosity with salt in a sulfated surfactant system, but there is no effect on viscosity with salt in a sulfate free surfactant system.

A further thickening behavior study was performed to determine viscosity differences with and without salt (e.g. sodium chloride). The results are shown in Table 18 below. Each column corresponds to a particular formulation with ingredients listed as a percentage by weight. The remainder was water, represented by Qs. % AM indicates % of active matter (e.g. % of dry surfactant). The viscosities for each formulation are bolded.

TABLE 18
Thickening behavior with and without salt

| 14% AM | Without Salt | | | With 1% NaCl | | |
|---|---|---|---|---|---|---|
| Aqua | 59.05 | 58.05 | 56.05 | 58.05 | 57.05 | 57.05 |
| Sodium Coco Sulfate | 8.00 | 8.00 | 8.00 | 8.00 | 8.00 | 8.00 |
| Guar | 0.25 | 0.25 | 0.25 | 0.25 | 0.25 | 0.25 |
| Hydroxypropyltrimonium | | | | | | |
| chloride | | | | | | |
| Decyl glucoside (50%) | 10 | 10 | 10 | 10 | 10 | 10 |
| Coco-Glucoside (and) | 3 | 3 | 3 | 3 | 3 | 3 |
| Glyceryl Oleate | | | | | | |
| Hydrolyzed Keratin | 1 | 1 | 1 | 1 | 1 | 1 |
| Sodium Chloride | 0 | 0 | 0 | 1 | 1 | 1 |
| 3-(heptyloxy)propane- | 0 | 1 | 1 | 0 | 1 | 1 |
| 1,2-diol | | | | | | |
| 3-[(n-octyl)oxy]-1,2- | 0 | 0 | 1 | 0 | 0 | 1 |
| propanediol | | | | | | |
| Citric Acid | pH 5.5 | pH 5.5 | pH 5.5 | pH 5.5 | pH 5.5 | pH 5.5 |
| Viscosity (LV3/12 rpm) | 70 cps | 80 cps | 380 cps | 280 cps | 1600 cps | 4380 cps |
| | | ≈ | ↑ | ↑ | ↑+ | ↑++ |

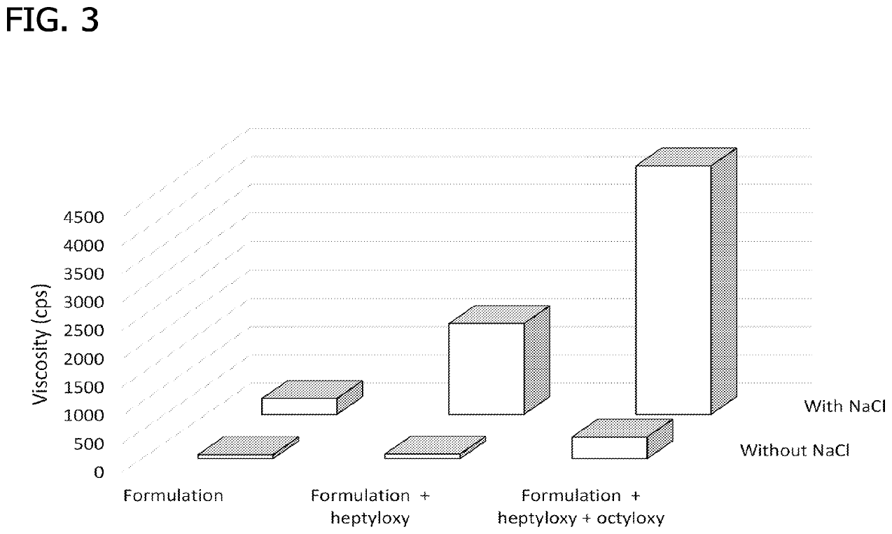

Table 18 demonstrates that the addition of 1% 3-(heptyloxy)propane-1,2-diol and 1% 3-[(n-octyl)oxy]-1,2-propanediol increases the viscosity of a salt free surfactant system. Addition of 1% 3-(heptyloxy)propane-1,2-diol and 1% 3-[(n-octyl)oxy]-1,2-propanediol considerably increases the viscosity of a salted surfactant system. A graphical representation of the viscosities in Table 18 can be found in FIG. 3.

In general, 3-(heptyloxy)propane-1,2-diol and 3-[(n-octyl)oxy]-1,2-propanediol are both demonstrated viscosity enhancers, particularly in combination with NaCl in sulfated surfactant systems. By using 3-(heptyloxy)propane-1,2-diol and 3-[(n-octyl)oxy]-1,2-propanediol together as viscosity enhancers in a basic sulfated surfactant system, only a small amount of salt is needed to obtain the optimal viscosity. This is ideal for lower irritation in formulations, as salt can cause dryness, irritation, and damage to hair.

### Example 6. Decreased Precipitation with 3-(heptyloxy)propane-1,2-diol and 3-[(n-octyl)oxy]-1,2-propanediol Premixing

Next the effect of premixing 3-(heptyloxy)propane-1,2-diol and 3-[(n-octyl)oxy]-1,2-propanediol before adding them to the composition compared to adding the compounds individually. Table 19 indicates the formulation used.

TABLE 19
Base Natural Shampoo Formulation for Premix Test

| Ingredient | % W/W | % AM |
|---|---|---|
| Aqua | Qs | |
| Sodium Coco Sulfate | 6 | 6 |
| Coco-Glucoside | 10 | 5 |
| Coco-Glucoside (and) Glyceryl | 3 | 1 |
| Oleate | | |
| Coco-Betaine | 5 | 1.5 |
| 3-(heptyloxy)propane-1,2-diol | 0.5 | |
| 3-[(n-octyl)oxy]-1,2-propanediol | 0.5 | |
| Aqua (and Citric Acid | Qs to pH 5.5 | |
| Sodium Chloride | 1 | |
| | | 13.5% AM |

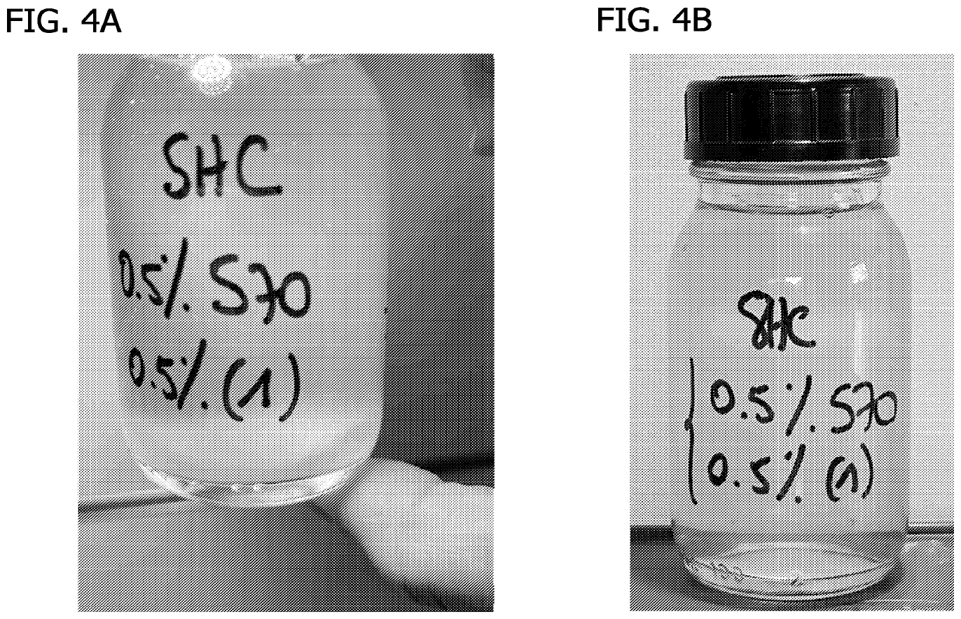

In the first process, the sodium coco-sulfate was dissolved into demineralized water heated to 65-70° C. The 3 other surfactants (sodium coco sulfate, coco-glucoside, and coco-glucoside with glyceryl oleate) were added into the blend while stirring. The mixture was further stirred until a homogeneous mixture was obtained. 3-(heptyloxy)propane-1,2-diol was added. 3-[(n-octyl)oxy]-1,2-propanediol was then added afterwards. The pH was adjusted to 5.5 with the citric acid solution. Sodium chloride was then added to adjust the viscosity. The resulting solution of this process is shown in FIG. 4A. A turbid solution with precipitation was observed.

The second process is identical to the first process except for the addition of 3-(heptyloxy)propane-1,2-diol and 3-[(n-octyl)oxy]-1,2-propanediol. The sodium coco-sulfate was dissolved into demineralized water heated to 65-70° C. The 3 other surfactants (sodium coco sulfate, coco-glucoside, and coco-glucoside with glyceryl oleate) were added into the blend while stirring. The mixture was further stirred until a homogeneous mixture was obtained. 3-(heptyloxy)propane-1,2-diol and 3-[(n-octyl)oxy]-1,2-propanediol were premixed and added into the main mixture already combined. The pH was adjusted to 5.5 with the citric acid solution. Sodium chloride was then added to adjust the viscosity. The result of this process is shown in FIG. 4B. A clear, homogeneous solution was observed.

Thus, premixing 3-(heptyloxy)propane-1,2-diol and 3-[(n-octyl)oxy]-1,2-propanediol enhances solubility and results in a clearer solution with less precipitate formed.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained. As various changes could be made in the above compositions and methods without departing from the scope of the invention, it is intended that all matter contained in the above description shall be interpreted as illustrative and not in a limiting sense.

1. A cosmetic composition comprising:
3-(heptyloxy)propane-1,2-diol;
3-[(n-octyl)oxy]-1,2-propanediol;
at least one surfactant; and
a rheology modifier comprising an inorganic salt.

2. The composition of claim 1, wherein the concentration of the inorganic salt to achieve a given viscosity is lower than an otherwise identical composition lacking 3-(heptyloxy)propane-1,2-diol and/or 3-[(n-octyl)oxy]-1,2-propanediol.

3. The composition of claim 1, wherein the at least one surfactant comprises an anionic surfactant, a non-ionic surfactant, an amphoteric surfactant, a cationic surfactant, a zwitterionic surfactant, or mixtures thereof.

4. The composition of claim 1, wherein the at least one surfactant is selected from the group consisting of an alkylpolyglucoside, sodium cocoylglutamate, sodium cocoylglycinate, sodium lauroyl sarcosinate, sodium lauryl sulfoacetate, lauryl glucose carboxylate, cocamidopropyl betaine, lauryl glucoside, decyl glucoside, caprylyl/capryl glucoside, coco-glucoside, hexyl glucoside, disodium cocoyl glutamate, sodium stearoyl glutamate, sodium palmoyl glutamate, sodium lauroyl glutamate, sodium myristoyl glutamate, potassium cocoyl glutamate, potassium lauroyl glutamate, potassium myristoyl glutamate, sodium lauroyl glycinate, potassium cocoyl glycinate, potassium cocoyl glycinate, sodium cocoyl isethionate, sodium cocoyl methyl isethionate, sodium lauroyl methyl isethionate, sodium methyl oleoyl taurate, sodium methyl lauroyl taurate, sodium methyl cocoyl taurate, cocoyl methyl glucamide, capryloyl/caproyl methyl glucamide, lauroyl/myristoyl methyl glucamide, cocobetaine, cocamidopropyl hydroxysultaine, lauryl betaine, sodium cocoamphoacetate, disodium cocoamphodiacetate, sodium lauroamphoacetate, sodium lauryl sulfoacetate, disodium laureth sulfosuccinate, disodium oleamido MIPA sulfosuccinate, cocamide DEA, cocamide MEA, cocamide MIPA, Sodium C14-16 olefin sulfonate, potassium cocoate, potassium palmate, potassium laurate, potassium olivate, sodium cocoyl sulfate, sodium lauryl sulfate, ammonium lauryl sulfate, sodium laureth sulfate, ammonium coco-sulfate, magnesium lauryl sulfate, magnesium laureth sulfate, ammonium laureth sulfate, sodium cetearyl sulfate, sodium myreth sulfate, magnesium myreth sulfate, ammonium myreth sulfate, sodium C₁₂ to C₁₈ alkyl sulfate, and mixtures thereof.

5. The composition of claim 1, wherein the at least one surfactant comprises an anionic surfactant, wherein the anionic surfactant comprises a sulfated surfactant.

6. The composition of claim 1, wherein the at least one surfactant comprises a mixture of at least one anionic surfactant and at least one non-ionic surfactant or a mixture of at least one anionic surfactant and at least one amphoteric surfactant.

7. The composition of claim 6, wherein the anionic surfactant comprises a sulfated surfactant, the non-ionic surfactant comprises an alkylpolyglucoside, and/or the amphoteric surfactant comprises a betaine.

8. (canceled)

9. The composition of claim 1, wherein the inorganic salt is selected from the group consisting of sodium chloride, ammonium chloride, and mixtures thereof.

10. (canceled)

11. The composition of claim 1, wherein the composition is at least 95% natural according to ISO standard 16128.

12. The composition of claim 1, wherein the composition further comprises at least one additional glycerol derivative.

13-14. (canceled)

15. The composition of claim 1, wherein the composition further comprises at least one additional preservative.

16.-17. (canceled)

18. The composition of claim 1, wherein the composition has a pH of from about 2 to about 10.5, from about 3 to about 8, from about 4 to about 7, from about 4 to about 6, from about 5 to about 7, from about 5.5 to about 7, from about 5.5 to about 6.5, or about 5.5.

19. The composition of claim 1, wherein the composition comprises a concentration of 3-(heptyloxy)propane-1,2-diol that is from about 0.1 wt. % to about 10 wt. %, from about 0.1 wt. % to about 3 wt. %, from about 0.1 wt. % to about 1.5 wt. %, from about 0.1 wt. % to about 1 wt. %, from about 0.25 wt. % to about 5 wt. %, from about 0.25 wt. % to about 3 wt. %, from about 0.25 wt. % to about 1.5 wt. %, from about 0.25 wt. % to about 1 wt. %, from about 0.5 wt. % to about 5 wt. %, from about 0.5 wt. % to about 3 wt. %, from about 0.5 wt. % to about 1.5 wt. %, from about 0.5 wt. % to about 1 wt. %, from about 1 wt. % to about 5 wt. %, from about 1 wt. % to about 3 wt. %, or from about 1 wt. % to about 1.5 wt. %.

20. The composition of claim 1, wherein the composition comprises a concentration of 3-[(n-octyl)oxy]-1,2-propanediol that is from about 0.1 wt. % to about 10 wt. %, from about 0.1 wt. % to about 3 wt. %, from about 0.1 wt. % to about 1.5 wt. %, from about 0.1 wt. % to about 1 wt. %, from about 0.25 wt. % to about 5 wt. %, from about 0.25 wt. % to about 3 wt. %, from about 0.25 wt. % to about 1.5 wt. %, from about 0.25 wt. % to about 1 wt. %, from about 0.5 wt. % to about 5 wt. %, from about 0.5 wt. % to about 3 wt. %, from about 0.5 wt. % to about 1.5 wt. %, from about 0.5 wt. % to about 1 wt. %, from about 1 wt. % to about 5 wt. %, from about 1 wt. % to about 3 wt. %, or from about 1 wt. % to about 1.5 wt. %.

21. The composition of claim 1, wherein the concentration of inorganic salt is from about from about 0.25 wt. % to about 5 wt. %, from about 0.5 wt. % to about 3 wt. %, or from about 1 wt. % to about 2 wt. %.

22. (canceled)

23. The composition of claim 1, wherein the concentration of the surfactant is from about 1% wt. % to about 5% wt. %.

24.-25. (canceled)

26. The composition of claim 1, wherein the composition is selected from the group consisting of shampoo, conditioner, dry shampoo, body wash, facial cleanser, face mask, bubble bath, cleansing milk, micellar water, make-up remover, cleansing wipes, hair mask, liquid soap, shaving soap, shaving foam, cleansing foam, day cream, anti-aging cream, body milk, facial lotion, body lotion, in-shower body lotion, body mousse, face serum, eye cream, sunscreen lotion, sun cream, face cream, after-shave lotion, pre-shaving cream, depilatory cream, skin-whitening gel, self-tanning cream, anti-acne gel, hair oil, hair styling gel, hair styling cream, pomade, anti-frizz serum, detangler, scalp treatment, hair colorant, split end fluid, deodorant, antiperspirant, baby cream, insect repellent, hand cream, sunscreen lotion, sunscreen gel, foot cream, exfoliator, body scrub, bar soap, hair treatment, mouthwash, toothpaste, teeth whitener, moisturizer, serum, toner, aqua sorbet, cream gel, styling mousse, hydro-alcoholic gel, body oil, shower milk, hair spray, combing cream, sunblock, fragrance, perfume, mascara, foundation, primer, concealer, blush, bronzer, setting powder, setting spray, blemish balm cream, color correcting cream, eyeliner, brow liner, night cream, eye brow gel, highlighter, lip stain, lip stick, lip gloss, lip balm, lip crayon, hand sanitizer, nail varnish remover, cellulite treatment, cuticle cream, eye shadow, bath additive, body mist, eau de toilette, lubricating gel, illuminator, hair spray, combing cream, baby powder, and diaper rash cream.

27. A shampoo composition comprising:
3-(heptyloxy)propane-1,2-diol;
3-[(n-octyl)oxy]-1,2-propanediol;
a surfactant selected from the group consisting of sodium cocoyl sulfate, sodium lauryl sulfate, ammonium lauryl sulfate, sodium laureth sulfate, ammonium coco-sulfate, magnesium lauryl sulfate, magnesium laureth sulfate, ammonium laureth sulfate, sodium cetearyl sulfate, sodium myreth sulfate, magnesium myreth sulfate, ammonium myreth sulfate, and sodium C₁₂ to C₁₈ alkyl sulfate; and
an optional rheology modifier comprising an inorganic salt;
wherein the concentration of the inorganic salt to achieve a given viscosity is lower than an otherwise identical composition lacking 3-(heptyloxy)propane-1,2-diol and/or 3-[(n-octyl)oxy]-1,2-propanediol.

28. The shampoo composition of claim 27, wherein the composition is free of a rheology modifier.

29. A method of producing a preserved cosmetic composition, the method comprising:
mixing 3-(heptyloxy)propane-1,2-diol and 3-[(n-octyl)oxy]-1,2-propanediol to form a preservative premix;
combining the preservative premix with the other components of the composition to form the preserved composition.

30.-34. (canceled)
